(19) United States
(12) Patent Application Publication
(10) Pub. No.: US 2003/0039973 A1
(43) Pub. Date: Feb. 27, 2003

(54) Human single nucleotide polymorphisms

(75) Inventors: Michele Cargill, Gaithersburg, MD (US); James S. Ireland, Gaithersburg, MD (US); Eric S. Lander, Cambridge, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA

(21) Appl. No.: 09/912,263

(22) Filed: Jul. 24, 2001

Related U.S. Application Data
(60) Provisional application No. 60/220,315, filed on Jul. 24, 2000.

(51) Int. Cl. C12Q 1/68; C07H 21/04; C12P 21/02; C12N 5/06

(52) U.S. Cl. 435/6; 435/69.1; 435/325; 435/320.1; 536/23.1

(57) ABSTRACT

The invention provides nucleic acid segments of the human genome, particularly nucleic acid segments from genes including polymorphic sites. Allele-specific primers and probes hybridizing to regions flanking or containing these sites are also provided. The nucleic acids, primers and probes are used in applications such as phenotype correlations, forensics, paternity testing, medicine and genetic analysis.

### RELATED APPLICATION

This application claims the benefit of U.S. Provisional Application No. 60/220,315 filed on Jul. 24, 2000, the entire teachings of which are incorporated herein by reference.

### BACKGROUND OF THE INVENTION

The genomes of all organisms undergo spontaneous mutation in the course of their continuing evolution, generating variant forms of progenitor nucleic acid sequences (Gusella, Ann. Rev. Biochem. 55, 831-854 (1986)). The variant form may confer an evolutionary advantage or disadvantage relative to a progenitor form, or may be neutral. In some instances, a variant form confers a lethal disadvantage and is not transmitted to subsequent generations of the organism. In other instances, a variant form confers an evolutionary advantage to the species and is eventually incorporated into the DNA of many or most members of the species and effectively becomes the progenitor form. In many instances, both progenitor and variant form(s) survive and co-exist in a species population. The coexistence of multiple forms of a sequence gives rise to polymorphisms.

Several different types of polymorphism have been reported. A restriction fragment length polymorphism (RFLP) is a variation in DNA sequence that alters the length of a restriction fragment (Botstein et al., Am. J Hum. Genet. 32, 314-331 (1980)). The restriction fragment length polymorphism may create or delete a restriction site, thus changing the length of the restriction fragment. RFLPs have been widely used in human and animal genetic analyses (see WO 90/13668; WO90/11369; Donis-Keller, Cell 51, 319-337 (1987); Lander et al., Genetics 121, 85-99 (1989)). When a heritable trait can be linked to a particular RFLP, the presence of the RFLP in an individual can be used to predict the likelihood that the animal will also exhibit the trait.

Other polymorphisms take the form of short tandem repeats (STRs) that include tandem di-, tri- and tetra-nucleotide repeated motifs. These tandem repeats are also referred to as variable number tandem repeat (VNTR) polymorphisms. VNTRs have been used in identity and paternity analysis (US 5,075,217; Armour et al., FEBS Lett. 307, 113-115 (1992); Horn et al., WO 91/14003; Jeffreys, EP 370,719), and in a large number of genetic mapping studies.

Other polymorphisms take the form of single nucleotide variations between individuals of the same species. Such polymorphisms are far more frequent than RFLPs, STRs and VNTRs. Some single nucleotide polymorphisms (SNP) occur in protein-coding nucleic acid sequences (coding sequence SNP (cSNP)), in which case, one of the polymorphic forms may give rise to the expression of a defective or otherwise variant protein and, potentially, a genetic disease. Examples of genes in which polymorphisms within coding sequences give rise to genetic disease include β-globin (sickle cell anemia), apoE4 (Alzheimer's Disease), Factor V Leiden (thrombosis), and CFTR (cystic fibrosis). cSNPs can alter the codon sequence of the gene and therefore specify an alternative amino acid. Such changes are called “missense” when another amino acid is substituted, and “nonsense” when the alternative codon specifies a stop signal in protein translation. When the cSNP does not alter the amino acid specified the cSNP is called “silent”.

Other single nucleotide polymorphisms occur in noncoding regions. Some of these polymorphisms may also result in defective protein expression (e.g., as a result of defective splicing). Other single nucleotide polymorphisms have no phenotypic effects.

Single nucleotide polymorphisms can be used in the same manner as RFLPs and VNTRs, but offer several advantages. Single nucleotide polymorphisms occur with greater frequency and are spaced more uniformly throughout the genome than other forms of polymorphism. The greater frequency and uniformity of single nucleotide polymorphisms means that there is a greater probability that such a polymorphism will be found in close proximity to a genetic locus of interest than would be the case for other polymorphisms. The different forms of characterized single nucleotide polymorphisms are often easier to distinguish than other types of polymorphism (e.g., by use of assays employing allele-specific hybridization probes or primers).

Only a small percentage of the total repository of polymorphisms in humans and other organisms has been identified. The limited number of polymorphisms identified to date is due to the large amount of work required for their detection by conventional methods. For example, a conventional approach to identifying polymorphisms might be to sequence the same stretch of DNA in a population of individuals by dideoxy sequencing. In this type of approach, the amount of work increases in proportion to both the length of sequence and the number of individuals in a population and becomes impractical for large stretches of DNA or large numbers of persons.

### SUMMARY OF THE INVENTION

Work described herein pertains to the identification of polymorphisms which can predispose individuals to disease, by resequencing large numbers of genes in a large number of individuals. Various genes from a number of individuals have been resequenced as described herein, and SNPs in these genes have been discovered (see the Table). Some of these SNPs are cSNPs which specify a different amino acid sequence (shown as mutation type “M” in the Table), some of the SNPs are silent cSNPs (shown as mutation type “S” in the Table), and some of these cSNPs specify a stop signal in protein translation (shown as an “N” in the “Mutation Type” column and an asterisk in the “Alt AA” column in the Table). Some of the identified SNPs were located in non-coding regions (indicated with a dash in the “Mutation Type” column in the Table).

The invention relates to a nucleic acid molecule which comprises a single nucleotide polymorphism at a specific location. In a particular embodiment the invention relates to the variant allele of a gene having a single nucleotide polymorphism, which variant allele differs from a reference allele by one nucleotide at the site(s) identified in the Table. Complements of these nucleic acid segments are also included. The segments can be DNA or RNA, and can be double- or single-stranded. Segments can be, for example, 5-10, 5-15, 10-20, 5-25, 10-30, 10-50 or 10-100 bases long.

The invention further provides allele-specific oligonucleotides that hybridize to a nucleic acid molecule comprising a single nucleotide polymorphism or to the complement of the nucleic acid molecule. These oligonucleotides can be probes or primers.

The invention further provides a method of analyzing a nucleic acid from an individual. The method allows the determination of whether the reference or variant base is present at any one of the polymorphic sites shown in the Table. Optionally, a set of bases occupying a set of the polymorphic sites shown in the Table is determined. This type of analysis can be performed on a number of individuals, who are also tested (previously, concurrently or subsequently) for the presence of a disease phenotype. The presence or absence of disease phenotype is then correlated with a base or set of bases present at the polymorphic site or sites in the individuals tested.

Thus, the invention further relates to a method of predicting the presence, absence, likelihood of the presence or absence, or severity of a particular phenotype or disorder associated with a particular genotype. The method comprises obtaining a nucleic acid sample from an individual and determining the identity of one or more bases (nucleotides) at specific (e.g., polymorphic) sites of nucleic acid molecules described herein, wherein the presence of a particular base at that site is correlated with a specified phenotype or disorder, thereby predicting the presence, absence, likelihood of the presence or absence, or severity of the phenotype or disorder in the individual.

The invention further relates to an oligonucleotide microarray having immobilized thereon a plurality of oligonucleotide probes specific for one or more nucleic acid molecules comprising a nucleic acid sequence selected from the group consisting of the nucleic acid sequences listed in the Table.

### DETAILED DESCRIPTION OF THE INVENTION

The present invention relates to a nucleic acid molecule which comprises a single nucleotide polymorphism (SNP) at a specific location. The nucleic acid molecule, e.g., a gene, which includes the SNP has at least two alleles, referred to herein as the reference allele and the variant allele. The reference allele (prototypical or wild type allele) has been designated arbitrarily and typically corresponds to the nucleotide sequence of the nucleic acid molecule which has been deposited with GenBank or TIGR under a given Accession number. The variant allele differs from the reference allele by one nucleotide at the site(s) identified in the Table. The present invention also relates to variant alleles of the described genes and to complements of the variant alleles. The invention further relates to portions of the variant alleles and portions of complements of the variant alleles which comprise (encompass) the site of the SNP and are at least 5 nucleotides in length. Portions can be, for example, 5-10, 5-15, 10-20, 5-25, 10-30, 10-50 or 10-100 bases long. For example, a portion of a variant allele which is 21 nucleotides in length includes the single nucleotide polymorphism (the nucleotide which differs from the reference allele at that site) and twenty additional nucleotides which flank the site in the variant allele. These additional nucleotides can be on one or both sides of the polymorphism. Polymorphisms which are the subject of this invention are defined in the Table with respect to the reference sequence deposited in GenBank or TIGR under the Accession number indicated.

For example, the invention relates to a portion of a gene (e.g., dopamine receptor D1 (DRD1)) having a nucleotide sequence as deposited in GenBank or TIGR (e.g., under Accession No. M67439) comprising a single nucleotide polymorphism at a specific position (e.g., nucleotide 861). The reference nucleotide for this polymorphic form of DRD1 is shown in column 8 of the Table, and the variant nucleotide is shown in column 9 of the Table. In a preferred embodiment, the nucleic acid molecule of the invention comprises the variant (alternate) nucleotide at the polymorphic position. For example, the invention relates to a nucleic acid molecule which comprises the nucleic acid sequence shown in row 1, column 6, of the Table having a “G” at nucleotide position 704. The nucleotide sequences of the invention can be double- or single-stranded.

The invention further provides allele-specific oligonucleotides that hybridize to a gene comprising a single nucleotide polymorphism or to the complement of the gene. Such oligonucleotides will hybridize to one polymorphic form of the nucleic acid molecules described herein but not to the other polymorphic form(s) of the sequence. Thus, such oligonucleotides can be used to determine the presence or absence of particular alleles of the polymorphic sequences described herein. These oligonucleotides can be probes or primers.

The invention further provides a method of analyzing a nucleic acid from an individual. The method determines which base is present at any one of the polymorphic sites shown in the Table. Optionally, a set of bases occupying a set of the polymorphic sites shown in the Table is determined. This type of analysis can be performed on a number of individuals, who are also tested (previously, concurrently or subsequently) for the presence of a disease phenotype. The presence or absence of disease phenotype is then correlated with a base or set of bases present at the polymorphic site or sites in the individuals tested.

Thus, the invention further relates to a method of predicting the presence, absence, likelihood of the presence or absence, or severity of a particular phenotype or disorder associated with a particular genotype. The method comprises obtaining a nucleic acid sample from an individual and determining the identity of one or more bases (nucleotides) at polymorphic sites of nucleic acid molecules described herein, wherein the presence of a particular base is correlated with a specified phenotype or disorder, thereby predicting the presence, absence, likelihood of the presence or absence, or severity of the phenotype or disorder in the individual. The correlation between a particular polymorphic form of a gene and a phenotype can thus be used in methods of diagnosis of that phenotype, as well as in the development of treatments for the phenotype.

Definitions

An oligonucleotide can be DNA or RNA, and single- or double-stranded. Oligonucleotides can be naturally occurring or synthetic, but are typically prepared by synthetic means. Preferred oligonucleotides of the invention include segments of DNA, or their complements, which include any one of the polymorphic sites shown in the Table. The segments can be between 5 and 250 bases, and, in specific embodiments, are between 5-10, 5-20, 10-20, 10-50, 20-50 or 10-100 bases. For example, the segment can be 21 bases. The polymorphic site can occur within any position of the segment. The segments can be from any of the allelic forms of DNA shown in the Table.

As used herein, the terms “nucleotide”, “base” and “nucleic acid” are intended to be equivalent. The terms “nucleotide sequence”, “nucleic acid sequence”, “nucleic acid molecule” and “segment” are intended to be equivalent.

Hybridization probes are oligonucleotides which bind in a base-specific manner to a complementary strand of nucleic acid. Such probes include peptide nucleic acids, as described in Nielsen et al., Science 254, 1497-1500 (1991). Probes can be any length suitable for specific hybridization to the target nucleic acid sequence. The most appropriate length of the probe may vary depending upon the hybridization method in which it is being used; for example, particular lengths may be more appropriate for use in microfabricated arrays, while other lengths may be more suitable for use in classical hybridization methods. Such optimizations are known to the skilled artisan. Suitable probes and primers can range from about 5 nucleotides to about 30 nucleotides in length. For example, probes and primers can be 5, 6, 8, 10, 12, 14, 16, 18, 20, 22, 24, 25, 26, 28 or 30 nucleotides in length. The probe or primer preferably overlaps at least one polymorphic site occupied by any of the possible variant nucleotides. The nucleotide sequence can correspond to the coding sequence of the allele or to the complement of the coding sequence of the allele.

As used herein, the term “primer” refers to a single-stranded oligonucleotide which acts as a point of initiation of template-directed DNA synthesis under appropriate conditions (e.g., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but typically ranges from 15 to 30 nucleotides. Short primer molecules generally require cooler temperatures to form sufficiently stable hybrid complexes with the template. A primer need not reflect the exact sequence of the template, but must be sufficiently complementary to hybridize with a template. The term primer site refers to the area of the target DNA to which a primer hybridizes. The term primer pair refers to a set of primers including a 5′ (upstream) primer that hybridizes with the 5′ end of the DNA sequence to be amplified and a 3′ (downstream) primer that hybridizes with the complement of the 3′ end of the sequence to be amplified.

As used herein, linkage describes the tendency of genes, alleles, loci or genetic markers to be inherited together as a result of their location on the same chromosome. It can be measured by percent recombination between the two genes, alleles, loci or genetic markers.

As used herein, polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic or biallelic polymorphism has two forms. A triallelic polymorphism has three forms.

Work described herein pertains to the resequencing of large numbers of genes in a large number of individuals to identify polymorphisms which can predispose individuals to disease. For example, polymorphisms in genes which are expressed in liver may predispose individuals to disorders of the liver.

By altering amino acid sequence, SNPs may alter the function of the encoded proteins. The discovery of the SNP facilitates biochemical analysis of the variants and the development of assays to characterize the variants and to screen for pharmaceutical that would interact directly with on or another form of the protein. SNPs (including silent SNPs) may also alter the regulation of the gene at the transcriptional or post-transcriptional level. SNPs (including silent SNPs) also enable the development of specific DNA, RNA, or protein-based diagnostics that detect the presence or absence of the polymorphism in particular conditions.

A single nucleotide polymorphism occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations).

A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. Typically the polymorphic site is occupied by a base other than the reference base. For example, where the reference allele contains the base “T” at the polymorphic site, the altered allele can contain a “C”, “G” or “A” at the polymorphic site.

Hybridizations are usually performed under stringent conditions, for example, at a salt concentration of no more than 1 M and a temperature of at least 25° C. For example, conditions of 5× SSPE (750 mM NaCl, 50 mM NaPhosphate, 5 mM EDTA, pH 7.4) and a temperature of 25-30° C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

The term “isolated” is used herein to indicate that the material in question exists in a physical milieu distinct from that in which it occurs in nature. For example, an isolated nucleic acid of the invention may be substantially isolated with respect to the complex cellular milieu in which it naturally occurs. In some instances, the isolated material will form part of a composition (for example, a crude extract containing other substances), buffer system or reagent mix. In other circumstance, the material may be purified to essential homogeneity, for example as determined by PAGE or column chromatography such as HPLC. Preferably, an isolated nucleic acid comprises at least about 50, 80 or 90 percent (on a molar basis) of all macromolecular species present.

I. Novel Polymorphisms of the Invention

The novel polymorphisms of the invention are shown in the Table. Columns one and two show designations for the indicated polymorphism. Column three shows the Genbank or TIGR Accession number for the wild type (or reference) allele. Column four shows the location (nucleotide position) of the polymorphic site in the nucleic acid sequence with reference to the Genbank or TIGR sequence shown in column three. Column five shows common names for the gene in which the polymorphism is located. Column six shows the polymorphism and a portion of the 3′ and 5′ flanking sequence of the gene. Column seven shows the type of mutation; N, non-sense; S, silent; and M, missense. Columns eight and nine show the reference and alternate nucleotides, respectively, at the polymorphic site. Columns ten and eleven show the reference and alternate amino acids, respectively, encoded by the reference and variant, respectively, alleles.

II. Analysis of Polymorphisms

A. Preparation of Samples

Polymorphisms are detected in a target nucleic acid from an individual being analyzed. For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed. For example, if the target nucleic acid is a cytochrome P450, the liver is a suitable source.

Many of the methods described below require amplification of DNA from target samples. This can be accomplished by e.g., PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

B. Detection of Polymorphisms in Target DNA

There are two distinct types of analysis of target DNA for detecting polymorphisms. The first type of analysis, sometimes referred to as de novo characterization, is carried out to identify polymorphic sites not previously characterized (i.e., to identify new polymorphisms). This analysis compares target sequences in different individuals to identify points of variation, i.e., polymorphic sites. By analyzing groups of individuals representing the greatest ethnic diversity among humans and greatest breed and species variety in plants and animals, patterns characteristic of the most common alleles/haplotypes of the locus can be identified, and the frequencies of such alleles/haplotypes in the population can be determined. Additional allelic frequencies can be determined for subpopulations characterized by criteria such as geography, race, or gender. The de novo identification of polymorphisms of the invention is described in the Examples section.

The second type of analysis determines which form(s) of a characterized (known) polymorphism are present in individuals under test. There are a variety of suitable procedures, including, but not limited to, those discussed below.

1. Allele-Specific Probes

The design and use of allele-specific probes for analyzing polymorphisms is described by e.g., Saiki et al., Nature 324, 163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15-mer at the 7 position; in a 16-mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

2. Tiling Arrays

The polymorphisms can also be identified by hybridization to nucleic acid arrays, some examples of which are described in WO 95/11995. The same arrays or different arrays can be used for analysis of characterized polymorphisms. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles as described, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

3. Allele-Specific Primers

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer which hybridizes at a distal site. Amplification proceeds from the two primers, resulting in a detectable product which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

4. Direct-Sequencing

The direct analysis of the sequence of polymorphisms of the present invention can be accomplished using either the dideoxy chain termination method or the Maxam—Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

5. Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applicationsfor DNA Amplification, (W.H. Freeman and Co, New York, 1992), Chapter 7.

6. Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

7. Single Base Extension

An alternative method for identifying and analyzing polymorphisms is based on single-base extension (SBE) of a fluorescently-labeled primer coupled with fluorescence resonance energy transfer (FRET) between the label of the added base and the label of the primer. Typically, the method, such as that described by Chen et al., (PNAS 94:10756-61 (1997)), uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently-labeled dideoxyribonucleotides (ddNTPs) in dye-terminator sequencing fashion. An increase in fluorescence of the added ddNTP in response to excitation at the wavelength of the labeled primer is used to infer the identity of the added nucleotide.

III. Methods of Use

The determination of the polymorphic form(s) present in an individual at one or more polymorphic sites defined herein can be used in a number of methods.

A. Forensics

Determination of which polymorphic forms occupy a set of polymorphic sites in an individual identifies a set of polymorphic forms that distinguishes the individual. See generally National Research Council, The Evaluation of Forensic DNA Evidence (Eds. Pollard et al., National Academy Press, DC, 1996). The more sites that are analyzed, the lower the probability that the set of polymorphic forms in one individual is the same as that in an unrelated individual. Preferably, if multiple sites are analyzed, the sites are unlinked. Thus, polymorphisms of the invention are often used in conjunction with polymorphisms in distal genes. Preferred polymorphisms for use in forensics are biallelic because the population frequencies of two polymorphic forms can usually be determined with greater accuracy than those of multiple polymorphic forms at multi-allelic loci.

The capacity to identify a distinguishing or unique set of forensic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers does match, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

p(ID) is the probability that two random individuals have the same polymorphic or allelic form at a given polymorphic site. In biallelic loci, four genotypes are possible: AA, AB, BA, and BB. If alleles A and B occur in a haploid genome of the organism with frequencies x and y, the probability of each genotype in a diploid organism is (see WO 95/12607):

Homozygote: p(AA)=x²

Homozygote: p(BB)=y²=(1−x)²

Single Heterozygote: p(AB)=p(BA)=xy=x(1−x)

Both Heterozygotes: p(AB+BA)=2xy=2x(1−x)

The probability of identity at one locus (i.e, the probability that two individuals, picked at random from a population will have identical polymorphic forms at a given locus) is given by the equation:

p(ID)=(x²)²+(2xy)²+(y²)².

These calculations can be extended for any number of polymorphic forms at a given locus. For example, the probability of identity p(ID) for a 3-allele system where the alleles have the frequencies in the population of x, y and z, respectively, is equal to the sum of the squares of the genotype frequencies:

p(ID)=x⁴+(2xy)²+(2yz)²+(2xz)²+z⁴+y⁴

In a locus of n alleles, the appropriate binomial expansion is used to calculate p(ID) and p(exc).

The cumulative probability of identity (cum p(ID)) for each of multiple unlinked loci is determined by multiplying the probabilities provided by each locus.

cum p(ID)=p(ID1)p(ID2)p(ID3) . . . p(IDn)

The cumulative probability of non-identity for n loci (i.e. the probability that two random individuals will be different at 1 or more loci) is given by the equation:

cum p(nonID)=1−cum p(ID).

If several polymorphic loci are tested, the cumulative probability of non-identity for random individuals becomes very high (e.g., one billion to one). Such probabilities can be taken into account together with other evidence in determining the guilt or innocence of the suspect.

B. Paternity Testing

The object of paternity testing is usually to determine whether a male is the father of a child. In most cases, the mother of the child is known and thus, the mother's contribution to the child's genotype can be traced. Paternity testing investigates whether the part of the child's genotype not attributable to the mother is consistent with that of the putative father. Paternity testing can be performed by analyzing sets of polymorphisms in the putative father and the child.

If the set of polymorphisms in the child attributable to the father does not match the set of polymorphisms of the putative father, it can be concluded, barring experimental error, that the putative father is not the real father. If the set of polymorphisms in the child attributable to the father does match the set of polymorphisms of the putative father, a statistical calculation can be performed to determine the probability of coincidental match.

The probability of parentage exclusion (representing the probability that a random male will have a polymorphic form at a given polymorphic site that makes him incompatible as the father) is given by the equation (see WO 95/12607):

p(exc)=xy(1−xy)

where x and y are the population frequencies of alleles A and B of a biallelic polymorphic site.

(At a triallelic site p(exc)=xy(1−xy)+yz(1−yz)+xz(1−xz)+3xyz(1−xyz))), where x, y and z and the respective population frequencies of alleles A, B and C).

The probability of non-exclusion is

p(non−exc)=1−p(exc)

The cumulative probability of non-exclusion (representing the value obtained when n loci are used) is thus:

cum p(non−exc)=p(non-exc1)p(non−exc2)p(non−exc3) . . . p(non−excn)

The cumulative probability of exclusion for n loci (representing the probability that a random male will be excluded)

cum p(exc)=1−cum p(non−exc).

If several polymorphic loci are included in the analysis, the cumulative probability of exclusion of a random male is very high. This probability can be taken into account in assessing the liability of a putative father whose polymorphic marker set matches the child's polymorphic marker set attributable to his/her father.

C. Correlation of Polymorphisms with Phenotypic Traits

The polymorphisms of the invention may contribute to the phenotype of an organism in different ways. Some polymorphisms occur within a protein coding sequence and contribute to phenotype by affecting protein structure. The effect may be neutral, beneficial or detrimental, or both beneficial and detrimental, depending on the circumstances. For example, a heterozygous sickle cell mutation confers resistance to malaria, but a homozygous sickle cell mutation is usually lethal. Other polymorphisms occur in noncoding regions but may exert phenotypic effects indirectly via influence on replication, transcription, and translation. A single polymorphism may affect more than one phenotypic trait. Likewise, a single phenotypic trait may be affected by polymorphisms in different genes. Further, some polymorphisms predispose an individual to a distinct mutation that is causally related to a certain phenotype.

Phenotypic traits include diseases that have known but hitherto unmapped genetic components (e.g., agammaglobulimenia, diabetes insipidus, Lesch-Nyhan syndrome, muscular dystrophy, Wiskott-Aldrich syndrome, Fabry's disease, familial hypercholesterolemia, polycystic kidney disease, hereditary spherocytosis, von Willebrand's disease, tuberous sclerosis, hereditary hemorrhagic telangiectasia, familial colonic polyposis, Ehlers-Danlos syndrome, osteogenesis imperfecta, and acute intermittent porphyria). Phenotypic traits also include symptoms of, or susceptibility to, multifactorial diseases of which a component is or may be genetic, such as autoimmune diseases, inflammation, cancer, diseases of the nervous system, and infection by pathogenic microorganisms. Some examples of autoimmune diseases include rheumatoid arthritis, multiple sclerosis, diabetes (insulin-dependent and non-independent), systemic lupus erythematosus and Graves disease. Some examples of cancers include cancers of the bladder, brain, breast, colon, esophagus, kidney, leukemia, liver, lung, oral cavity, ovary, pancreas, prostate, skin, stomach and uterus. Phenotypic traits also include characteristics such as longevity, appearance (e.g., baldness, obesity), strength, speed, endurance, fertility, and susceptibility or receptivity to particular drugs or therapeutic treatments.

The correlation of one or more polymorphisms with phenotypic traits can be facilitated by knowledge of the gene product of the wild type (reference) gene. The genes in which SNPs of the present invention have been identified are genes which have been previously sequenced and characterized in one of their allelic forms. Thus, the SNPs of the invention can be used to identify correlations between one or another allelic form of the gene with a disorder with which the gene is associated, thereby identifying causative or predictive allelic forms of the gene.

Correlation is performed for a population of individuals who have been tested for the presence or absence of a phenotypic trait of interest and for polymorphic markers sets. To perform such analysis, the presence or absence of a set of polymorphisms (i.e. a polymorphic set) is determined for a set of the individuals, some of whom exhibit a particular trait, and some of which exhibit lack of the trait. The alleles of each polymorphism of the set are then reviewed to determine whether the presence or absence of a particular allele is associated with the trait of interest. Correlation can be performed by standard statistical methods such as a κ-squared test and statistically significant correlations between polymorphic form(s) and phenotypic characteristics are noted. For example, it might be found that the presence of allele A1 at polymorphism A correlates with heart disease. As a further example, it might be found that the combined presence of allele A1 at polymorphism A and allele B1 at polymorphism B correlates with increased milk production of a farm animal.

Such correlations can be exploited in several ways. In the case of a strong correlation between a set of one or more polymorphic forms and a disease for which treatment is available, detection of the polymorphic form set in a human or animal patient may justify immediate administration of treatment, or at least the institution of regular monitoring of the patient. Detection of a polymorphic form correlated with serious disease in a couple contemplating a family may also be valuable to the couple in their reproductive decisions. For example, the female partner might elect to undergo in vitro fertilization to avoid the possibility of transmitting such a polymorphism from her husband to her offspring. In the case of a weaker, but still statistically significant correlation between a polymorphic set and human disease, immediate therapeutic intervention or monitoring may not be justified. Nevertheless, the patient can be motivated to begin simple life-style changes (e.g., diet, exercise) that can be accomplished at little cost to the patient but confer potential benefits in reducing the risk of conditions to which the patient may have increased susceptibility by virtue of variant alleles. Identification of a polymorphic set in a patient correlated with enhanced receptiveness to one of several treatment regimes for a disease indicates that this treatment regime should be followed.

For animals and plants, correlations between characteristics and phenotype are useful for breeding for desired characteristics. For example, Beitz et al., U.S. Pat. No. 5,292,639 discuss use of bovine mitochondrial polymorphisms in a breeding program to improve milk production in cows. To evaluate the effect of mtDNA D-loop sequence polymorphism on milk production, each cow was assigned a value of 1 if variant or 0 if wildtype with respect to a prototypical mitochondrial DNA sequence at each of 17 locations considered. Each production trait was analyzed individually with the following animal model:

Yᵢⱼₖₚₙ=μ+YSᵢ+Pⱼ+Xₖ+βᵢ+ . . . β₁₇+PEₙ+aₙ+eₚ

where Yᵢⱼₖₙₚ is the milk, fat, fat percentage, SNF, SNF percentage, energy concentration, or lactation energy record; μ is an overall mean; YSᵢ is the effect common to all cows calving in year-season; Xₖ is the effect common to cows in either the high or average selection line; β₁ to β₁₇ are the binomial regressions of production record on mtDNA D-loop sequence polymorphisms; PEₙ is permanent environmental effect common to all records of cow n; aₙ is effect of animal n and is composed of the additive genetic contribution of sire and dam breeding values and a Mendelian sampling effect; and eₚ is a random residual. It was found that eleven of seventeen polymorphisms tested influenced at least one production trait. Bovines having the best polymorphic forms for milk production at these eleven loci are used as parents for breeding the next generation of the herd.

D. Genetic Mapping of Phenotypic Traits

The previous section concerns identifying correlations between phenotypic traits and polymorphisms that directly or indirectly contribute to those traits. The present section describes identification of a physical linkage between a genetic locus associated with a trait of interest and polymorphic markers that are not associated with the trait, but are in physical proximity with the genetic locus responsible for the trait and co-segregate with it. Such analysis is useful for mapping a genetic locus associated with a phenotypic trait to a chromosomal position, and thereby cloning gene(s) responsible for the trait. See Lander et al., Proc. Natl. Acad. Sci. (USA) 83, 7353-7357 (1986); Lander et al., Proc. Natl. Acad. Sci. (USA) 84, 2363-2367 (1987); Donis-Keller et al., Cell 51, 319-337 (1987); Lander et al., Genetics 121, 185-199 (1989)). Genes localized by linkage can be cloned by a process known as directional cloning. See Wainwright, Med. J. Australia 159, 170-174 (1993); Collins, Nature Genetics 1, 3-6 (1992).

Linkage studies are typically performed on members of a family. Available members of the family are characterized for the presence or absence of a phenotypic trait and for a set of polymorphic markers. The distribution of polymorphic markers in an informative meiosis is then analyzed to determine which polymorphic markers co-segregate with a phenotypic trait. See, e.g., Kerem et al., Science 245, 1073-1080 (1989); Monaco et al., Nature 316, 842 (1985); Yamoka et al., Neurology 40, 222-226 (1990); Rossiter et al., FASEB Journal 5, 21-27 (1991).

Linkage is analyzed by calculation of LOD (log of the odds) values. A lod value is the relative likelihood of obtaining observed segregation data for a marker and a genetic locus when the two are located at a recombination fraction θ, versus the situation in which the two are not linked, and thus segregating independently (Thompson & Thompson, Genetics in Medicine (5th ed, W.B. Saunders Company, Philadelphia, 1991); Strachan, “Mapping the human genome” in The Human Genome (BIOS Scientific Publishers Ltd, Oxford), Chapter 4). A series of likelihood ratios are calculated at various recombination fractions (θ), ranging from θ=0.0 (coincident loci) to θ=0.50 (unlinked). Thus, the likelihood at a given value of θ is: probability of data if loci linked at θ to probability of data if loci unlinked. The computed likelihoods are usually expressed as the log₁₀ of this ratio (i.e., a lod score). For example, a lod score of 3 indicates 1000:1 odds against an apparent observed linkage being a coincidence. The use of logarithms allows data collected from different families to be combined by simple addition. Computer programs are available for the calculation of lod scores for differing values of θ (e.g., LIPED, MLINK (Lathrop, Proc. Nat. Acad. Sci. (USA) 81, 3443-3446 (1984)). For any particular lod score, a recombination fraction may be determined from mathematical tables. See Smith et al., Mathematical tables for research workers in human genetics (Churchill, London, 1961); Smith, Ann. Hum. Genet. 32, 127-150 (1968). The value of θ at which the lod score is the highest is considered to be the best estimate of the recombination fraction.

Positive lod score values suggest that the two loci are linked, whereas negative values suggest that linkage is less likely (at that value of θ) than the possibility that the two loci are unlinked. By convention, a combined lod score of +3 or greater (equivalent to greater than 1000:1 odds in favor of linkage) is considered definitive evidence that two loci are linked. Similarly, by convention, a negative lod score of −2 or less is taken as definitive evidence against linkage of the two loci being compared. Negative linkage data are useful in excluding a chromosome or a segment thereof from consideration. The search focuses on the remaining non-excluded chromosomal locations.

IV. Modified Polypeptides and Gene Sequences

The invention further provides variant forms of nucleic acids and corresponding proteins. The nucleic acids comprise one of the sequences described in the Table, column 5, in which the polymorphic position is occupied by one of the alternative bases for that position. Some nucleic acids encode full-length variant forms of proteins. Similarly, variant proteins have the prototypical amino acid sequences encoded by nucleic acid sequences shown in the Table, column 6, (read so as to be in-frame with the full-length coding sequence of which it is a component) except at an amino acid encoded by a codon including one of the polymorphic positions shown in the Table. That position is occupied by the variant or alternative amino acid shown in the Table.

Variant genes can be expressed in an expression vector in which a variant gene is operably linked to a native or other promoter. Usually, the promoter is a eukaryotic promoter for expression in a mammalian cell. The transcription regulation sequences typically include a heterologous promoter and optionally an enhancer which is recognized by the host. The selection of an appropriate promoter, for example trp, lac, phage promoters, glycolytic enzyme promoters and tRNA promoters, depends on the host selected. Commercially available expression vectors can be used. Vectors can include host-recognized replication systems, amplifiable genes, selectable markers, host sequences useful for insertion into the host genome, and the like.

The means of introducing the expression construct into a host cell varies depending upon the particular construction and the target host. Suitable means include fusion, conjugation, transfection, transduction, electroporation or injection, as described in Sambrook, supra. A wide variety of host cells can be employed for expression of the variant gene, both prokaryotic and eukaryotic. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the variant gene product to produce an appropriate mature polypeptide. Processing includes glycosylation, ubiquitination, disulfide bond formation, general post-translational modification, and the like. As used herein, “gene product” includes mRNA, peptide and protein products.

The protein may be isolated by conventional means of protein biochemistry and purification to obtain a substantially pure product, i.e., 80, 95 or 99% free of cell component contaminants, as described in Jacoby, Methods in Enzymology Volume 104, Academic Press, New York (1984); Scopes, Protein Purification, Principles and Practice, 2nd Edition, Springer-Verlag, New York (1987); and Deutscher (ed), Guide to Protein Purification, Methods in Enzymology, Vol. 182 (1990). If the protein is secreted, it can be isolated from the supernatant in which the host cell is grown. If not secreted, the protein can be isolated from a lysate of the host cells.

The invention further provides transgenic nonhuman animals capable of expressing an exogenous variant gene and/or having one or both alleles of an endogenous variant gene inactivated. Expression of an exogenous variant gene is usually achieved by operably linking the gene to a promoter and optionally an enhancer, and microinjecting the construct into a zygote. See Hogan et al., “Manipulating the Mouse Embryo, A Laboratory Manual,” Cold Spring Harbor Laboratory. Inactivation of endogenous variant genes can be achieved by forming a transgene in which a cloned variant gene is inactivated by insertion of a positive selection marker. See Capecchi, Science 244, 1288-1292 (1989). The transgene is then introduced into an embryonic stem cell, where it undergoes homologous recombination with an endogenous variant gene. Mice and other rodents are preferred animals. Such animals provide useful drug screening systems.

In addition to substantially fiull-length polypeptides expressed by variant genes, the present invention includes biologically active fragments of the polypeptides, or analogs thereof, including organic molecules which simulate the interactions of the peptides. Biologically active fragments include any portion of the full-length polypeptide which confers a biological function on the variant gene product, including ligand binding, and antibody binding. Ligand binding includes binding by nucleic acids, proteins or polypeptides, small biologically active molecules, or large cellular structures.

Polyclonal and/or monoclonal antibodies that specifically bind to variant gene products but not to corresponding prototypical gene products are also provided. Antibodies can be made by injecting mice or other animals with the variant gene product or synthetic peptide fragments thereof. Monoclonal antibodies are screened as are described, for example, in Harlow & Lane, Antibodies, A Laboratory Manual, Cold Spring Harbor Press, New York (1988); Goding, Monoclonal antibodies, Principles and Practice (2d ed.) Academic Press, New York (1986). Monoclonal antibodies are tested for specific immunoreactivity with a variant gene product and lack of immunoreactivity to the corresponding prototypical gene product. These antibodies are useful in diagnostic assays for detection of the variant form, or as an active ingredient in a pharmaceutical composition.

V. Kits

The invention further provides kits comprising at least one agent for identifying which alleleic form of the SNPs identified herein is present in a sample. For example, suitable kits can comprise at least one antibody specific for a particular protein or peptide encoded by one alleleic form of the gene, or allele-specific oligonucleotide as described herein. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting at least 10, 100 or all of the polymorphisms shown in the Table. Optional additional components of the kit include, for example, restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, the kit also contains instructions for carrying out the methods.

The following Examples are offered for the purpose of illustrating the present invention and are not to be construed to limit the scope of this invention. The teachings of all references cited herein are hereby incorporated herein by reference.

### EXAMPLES

The polymorphisms shown in the Table were identified by resequencing of target sequences from individuals of diverse ethnic and geographic backgrounds by hybridization to probes immobilized to microfabricated arrays. The strategy and principles for design and use of such arrays are generally described in WO 95/11995. Accordingly, the invention encompasses an oligonucleotide microarray having immobilized thereon a plurality of oligonucleotide probes specific for one or more nucleic acid molecules comprising a nucleic acid sequence selected from the group consisting of the nucleic acid sequences listed in the Table.

A typical probe array used in this analysis has two groups of four sets of probes that respectively tile both strands of a reference sequence. A first probe set comprises a plurality of probes exhibiting perfect complementarily with one of the reference sequences. Each probe in the first probe set has an interrogation position that corresponds to a nucleotide in the reference sequence. That is, the interrogation position is aligned with the corresponding nucleotide in the reference sequence, when the probe and reference sequence are aligned to maximize complementarily between the two. For each probe in the first set, there are three corresponding probes from three additional probe sets. Thus, there are four probes corresponding to each nucleotide in the reference sequence. The probes from the three additional probe sets are identical to the corresponding probe from the first probe set except at the interrogation position, which occurs in the same position in each of the four corresponding probes from the four probe sets, and is occupied by a different nucleotide in the four probe sets. In the present analysis, probes were 25 nucleotides long. Arrays tiled for multiple different references sequences were included on the same substrate.

Publicly available sequences for a given gene were assembled into Gap4 (http://www.biozentrum.unibas.ch/˜biocomp/staden/Overview.html). PCR primers covering each exon were designed using Primer 3 (http://www-genome.wi.mit.edu/cgi-bin/primer/primer3.cgi). Primers were not designed in regions where there were sequence discrepancies between reads. Genomic DNA was amplified in at least 50 individuals using 2.5 pmol each primer, 1.5 mM MgCl₂, 100 μM dNTPs, 0.75 μM AmpliTaq GOLD polymerase, and 19 ng DNA in a 15 μl reaction. Reactions were assembled using a PACKARD MultiPROBE robotic pipetting station and then put in MJ 96-well tetrad thermocyclers (96° C. for 10 minutes, followed by 35 cycles of 96° C. for 30 seconds, 59° C. for 2 minutes, and 72° C. for 2 minutes). A subset of the PCR assays for each individual were run on 3% NuSieve gels in 0.5X TBE to confirm that the reaction worked.

For a given DNA, 5 μl (about 50 ng) of each PCR or RT-PCR product were pooled (Final volume=150-200 μl). The products were purified using QiaQuick PCR purification from Qiagen. The samples were eluted once in 35 μl sterile water and 4 μl 10× One-Phor-All buffer (Pharmacia). The pooled samples were digested with 0.2μ DNaseI (Promega) for 10 minutes at 37° C. and then labeled with 0.5 nmols biotin-N6-ddATP and 15μ Terminal Transferase (GibcoBRL Life Technology) for 60 minutes at 37° C. Both fragmentation and labeling reactions were terminated by incubating the pooled sample for 15 minutes at 100° C.

Low-density DNA chips (Affymetrix, Calif.) were hybridized following the manufacturer's instructions. Briefly, the hybridization cocktail consisted of 3M TMACl, 10 mM Tris pH 7.8, 0.01% Triton X-100, 100 mg/ml herring sperm DNA (Gibco BRL), 200 pM control biotin-labeled oligo. The processed PCR products were denatured for 7 minutes at 100° C. and then added to prewarmed (37° C.) hybridization solution. The chips were hybridized overnight at 44° C. Chips were washed in 1× SSPET and 6× SSPET followed by staining with 2 μg/ml SARPE and 0.5 mg/ml acetylated BSA in 200 μl of 6× SSPET for 8 minutes at room temperature. Chips were scanned using a Molecular Dynamics scanner.

Chip image files were analyzed using Ulysses (Affymetrix, Calif.) which uses four algorithms to identify potential polymorphisms. Candidate polymorphisms were visually inspected and assigned a confidence value: high confidence candidates displayed all three genotypes, while likely candidates showed only two genotypes (homozygous for reference sequence and heterozygous for reference and variant). Some of the candidate polymorphisms were confirmed by ABI sequencing. Identified polymorphisms were compared to several databases to determine if they were novel. Results are shown in the Table.

| | | Genbank or TIGR | Position in | | | Mutation | Ref | Alt | Ref | Alt |
|---|---|---|---|---|---|---|---|---|---|---|
| Poly ID | WIAF ID | Accession Number | Sequence | Gene Description | Flanking Seq | Type | NT | NT | AA | AA |
| DRD5a64 | WI-18269 | M67439 | 861 | DRD1, dopamine receptor D1 | CTTCTACATCCCCGT[T/G]GCCATCATGATCGTG | S | T | G | V | V |
| DRD5a65 | WI-18270 | M67439 | 916 | DRD1, dopamine receptor D1 | GCCCAGGTGCAGATC[C/T]GCAGGATTTCCTCCC | M | C | T | R | C |
| DRD5a66 | WI-18271 | M67439 | 982 | DRD1, dopamine receptor D1 | AGCAGCGCAGCCTGC[G/A]CGCCCGACACCAGCC | M | G | A | A | T |
| G10a4 | WI-18661 | J04111 | 1366 | JUN, v-jun avian sarcoma | CCCAAGATCCTGAAA[C/T]AGAGCATGACCCTGA | N | C | T | Q | * |
| | | | | virus 17 oncogene homolog | | | | | | |
| G10a5 | WI-18662 | J04111 | 1595 | JUN, v-jun avian sarcoma | AGGAGGGGTTCGCCG[A/G]GGGCTTCGTGCGCGC | M | A | G | E | G |
| | | | | virus 17 oncogene homolog | | | | | | |
| G1041a4 | WI-18958 | X72886 | 451 | H. sapiens TYRO3 mRNA., ? | GCAGAGGACATGACA[G/T]TGTGTGTGGCTGACT | M | G | T | V | L |
| G1092a1 | WI-18960 | HT0547 | 1703 | KCNC4, potassium voltage- | AAGAGACTTCCCCCC[G/A]GGACAGCACCTGCAG | M | G | A | R | Q |
| | | | | gated channel, Shaw-related | | | | | | |
| | | | | subfamily, member 4 | | | | | | |
| G1098a14 | WI-19491 | L19711 | 2723 | DAG1, dystroglycan 1 | ATGATCTGCTACCGC[A/C]AGAAGCGGAAGGGCA | M | A | C | K | Q |
| | | | | (dystrophin-associated | | | | | | |
| | | | | glycoprotein 1) | | | | | | |
| G1098a15 | WI-19492 | L19711 | 2758 | DAG1,dystroglycan 1 | TACCCTTGAGGACCA[G/A]GCCACCTTCATCAAG | S | G | A | Q | Q |
| | | | | (dystrophin-associated | | | | | | |
| | | | | glycoprotein 1) | | | | | | |
| G1124a1 | WI-18959 | HT0367 | 168 | peripherin, ? | GAGCTCCTCGGTGCG[C/T]CTGGGCAGCTTCCGT | S | C | T | R | R |
| G1461a2 | WI-18465 | HT0329 | 1312 | pRB-binding protein, ? | CGGGAGGACTTCTCC[G/A]GCCTCCTCCCTGAGG | M | G | A | G | S |
| G1461a3 | WI-18466 | HT0329 | 1514 | pRB-binding protein, ? | AGCCCTGGAGCCCCC[T/C]GTCCCTGGCCGTCCT | - | T | C | | |
| G1461a4 | WI-18799 | HT0329 | 876 | pRB-binding protein, ? | CCTGGCCTACGTGAC[G/A]TGTCAGGACCTTCGT | S | G | A | T | T |
| G1468a4 | WI-18462 | HT4968 | 1409 | apoptosis inhibitor, | CGATCACACCAGATG[T/C]TTTCCCAATTGTCCA | S | T | C | C | C |
| | | | | neuronal, ? | | | | | | |
| G1468a5 | WI-18463 | HT4986 | 1998 | apoptosis inhibitor, | GTTACTGAAATGTGC[A/G]TGAGGAACATTATCC | M | A | G | M | V |
| | | | | neuronal, | | | | | | |
| G1468a6 | WI-18464 | HT4986 | 2275 | apoptosis inhibitor, | TGCGAAAGTTTATGG[T/G]TTACTTTGGAAAGAA | M | T | G | V | G |
| | | | | neuronal, ? | | | | | | |
| G1479a11 | WI-18964 | Y09077 | 7570 | ATR, ataxia telangiectasia | TGTAGATTTCAATTG[T/C]CTTTTCAATAAGGGA | S | T | C | C | C |
| | | | | and Rad3 related | | | | | | |
| G1480a2 | WI-18800 | HT1406 | 841 | G22P1, thyroid autoantigen | GTGATCTCTGTGGGC[A/G]TTTATAATCTGGTCC | M | A | G | I | V |
| | | | | 70kD (Ku antigen) | | | | | | |
| G1485a6 | WI-18798 | HT1432 | 3289 | BCR, breakpoint cluster | AAAGCAAAGACGCGC[G/A]TCTACAGGGACACAG | M | G | A | V | I |
| | | | | region | | | | | | |
| G1492a11 | WI-18249 | HT3506 | 558 | cell death-associated | GGAGATCCAGCACCC[C/T]AATGTCATCACCCTG | S | C | T | P | P |
| | | | | kinase, ? | | | | | | |
| G1500a1 | WI-18467 | HT2519 | 293 | CSF2, colony stimulating | CCTGCGGGGCAGCCT[C/T]ACCAAGCTCAAGGGC | S | C | T | L | L |
| | | | | factor 2 (granulocyte- | | | | | | |
| | | | | macrophage) | | | | | | |
| G1500a2 | WI-18468 | HT2519 | 382 | CSF2, colony stimulating | CCTGTGCAACCCAGA[T/C]TATCACCTTTGAAAG | M | T | C | I | T |
| | | | | factor 2 (granulocyte- | | | | | | |
| | | | | macrophage) | | | | | | |
| G1501a7 | WI-18470 | HT1949 | 494 | MCC, mutated in colorectal | AGCGTCATTGCGGAG[C/T]TCAACAAGAAGATAG | M | C | T | L | F |
| | | | | cancers | | | | | | |
| G1501a8 | WI-18471 | HT1949 | 1500 | MCC, mutated in colorectal | AGGAATGTAAAAGCA[A/T]TGCTGAGAGGATGAG | M | A | T | N | I |
| | | | | cancers | | | | | | |
| G1501a9 | WI-18472 | HT1949 | 2293 | MCC, mutated in colorectal | CAGCGGCAGCAAAGA[T/C]AAACCTGGCAAGGAG | S | T | C | D | D |
| | | | | cancers | | | | | | |
| G1502a3 | WI-18251 | HT1547 | 1006 | CCND1 cyclin D1 (PRAD1: | GACCTGGCTTGCACA[C/T]CCACCGACGTGCGGG | M | C | T | P | S |
| | | | | parathyroid adenomatosis 1) | | | | | | |
| G1515a3 | WI-18250 | HT2912 | 1137 | CDH1, cadherin 1, E- | TGGTGGTTCAAGCTG[C/A]TGACCTTCAAGGTGA | M | C | A | A | D |
| | | | | cadherin (epithelial) | | | | | | |
| G1517a13 | WI-19556 | HT1132 | 3389 | ERBB3, v-erb-b2 avian | AGGGTAATCTTGGGG[G/A]GTCTTGCCAGGAGTC | M | G | A | G | E |
| | | | | erythroblastic leukemia | | | | | | |
| | | | | viral oncogene homolog 3 | | | | | | |
| G1517a14 | WI-19557 | HT1132 | 3546 | ERBB3, v-erb-b2 avian | AGTGTCAATGTGTAG[A/G]AGCCGGAGCAGGAGC | S | A | G | R | R |
| | | | | erythroblastic leukemia | | | | | | |
| | | | | viral oncogene homolog 3 | | | | | | |
| G1517a15 | WI-19558 | HT1132 | 4280 | ERBB3, v-erb-b2 avian | CAGCTAGTGCCTTTA[G/A]AGGGTACCGTCTTCT | - | G | A | | |
| | | | | erythroblastic leukemia | | | | | | |
| | | | | viral oncogene homolog 3 | | | | | | |
| G1520a3 | WI-18672 | HT1175 | 730 | DNA excision repair protein | CTGGACCCCAAGATT[G/A]CAGACCTGGTGTCCA | M | G | A | A | T |
| | | | | ERCC2, 5′ end, ? | | | | | | |
| G1520a4 | WI-19281 | HT1175 | 1012 | DNA excision repair protein | AACCCCGTGCTGCCC[G/A]ACGAAGTGCTGCAGG | M | G | A | D | N |
| | | | | ERCC2, 5′ end, ? | | | | | | |
| G154a7 | WI-19330 | HT2645 | 2868 | proto-oncogene c-kit, alt. | CAACCGACAGAAGCC[C/T]GTGGTAGACCATTCT | M | C | T | | A |
| | | | | transcript 1, ? | | | | | | |
| G1572a6 | WI-18670 | HT3998 | 2296 | proto-oncogene c-abl, | GGAGCGCAGAGGGGC[C/T]GGCGAGGAAGAGGGC | S | C | T | A | A |
| | | | | tyrosine protein kinase, | | | | | | |
| | | | | alt. transcript 2, ? | | | | | | |
| G1572a7 | WI-18673 | HT3998 | 1117 | proto-oncogene c-abl, | GGAGATGGAACGCAC[G/A]GACATCACCATGAAG | S | G | A | T | T |
| | | | | tyrosine protein kinase, | | | | | | |
| | | | | alt. transcript 2, ? | | | | | | |
| G1572a8 | WI-18674 | HT3998 | 2749 | proto-oncogene c-abl, | AGTAACGCCTCCCCC[C/G]AGGCTGGTGAAAAAG | S | C | G | P | P |
| | | | | tyrosine protein kinase, | | | | | | |
| | | | | alt. transcript 2, ? | | | | | | |
| G1572a9 | WI-18675 | HT3998 | 2826 | proto-oncogene c-abl, | GCCCGGGCTCCAGCC[C/T]GCCCAACCTGACTCC | M | C | T | P | L |
| | | | | tyrosine protein kinase, | | | | | | |
| | | | | alt. transcript 2, ? | | | | | | |
| G1572a10 | WI-18676 | HT3998 | 3859 | proto-oncogene c-abl, | GGCTCGCCCATACCC[G/A]TGACAGTGGCTGACA | - | G | A | | |
| | | | | tyrosine protein kinase, | | | | | | |
| | | | | alt. transcript 2, ? | | | | | | |
| G1573a13 | WI-18252 | HT0642 | 300 | CBL,Cas-Br-M (murine) | ACCCGCCGGGGACGG[T/C]GGACAAGAAGATGGT | M | T | C | V | A |
| | | | | ecotropic retroviral | | | | | | |
| | | | | transforming sequence | | | | | | |
| G1574a10 | WI-18253 | HT1508 | 1461 | FES, feline sarcoma (Snyder- | GCAGCTGTGGTACCA[C/T]GGGGCCATCCCGAGG | S | C | T | H | H |
| | | | | Theilen) viral (v- | | | | | | |
| | | | | fes)/Fujinami avian sarcoma | | | | | | |
| | | | | (PRCII) viral (v-fps) | | | | | | |
| | | | | oncogene homolog | | | | | | |
| G1568a1 | WI-18259 | HT2291 | 465 | SRC, v-src avian sarcoma | GTGGTATTTTGGCAA[G/A]ATCACCAGACGGGAG | S | G | A | K | K |
| | | | | (Schmidt-Ruppin A-2) viral | | | | | | |
| | | | | oncogene homolog | | | | | | |
| G1587a11 | WI-18254 | HT0590 | 1536 | proto-oncogene dbl, ? | TTTTTCATCTAAACA[A/G]GGGAAGAAGACTTGG | S | A | G | Q | Q |
| G159a2 | WI-18458 | HT4209 | 1155 | RAD23B, RAD23 (S. | TTGAATTTTTACGGA[A/T]TCAGCCTCAGTTTCA | M | A | T | N | I |
| | | | | cerevisiae) homolog B | | | | | | |
| G159a3 | WI-18659 | HT4209 | 1415 | RAD23B,RAD23 (S. | ACACCTCAGGAAAA[G/A]AAGCTATAGAAAGGT | M | G | A | E | K |
| | | | | cerevisiae) homolog B | | | | | | |
| G159a4 | WI-18660 | HT4209 | 1474 | RAD23B, RAD23 (S. | TGTGATACAAGCGTA[T/C]TTTGCTTGTGAGAAG | S | T | C | Y | Y |
| | | | | cerevisiae) homolog B | | | | | | |
| G1602a2 | WI-18260 | HT1903 | 1426 | proto-oncogene pim-1, ? | CCAGTGACACGTCTC[G/T]CCAAGCAGGACAGTG | - | G | T | | |
| G1602a3 | WI-18261 | HT1903 | 1427 | proto-oncogene pim-1, ? | CAGTGACACGTCTCG[C/A]CAAGCAGGACAGTGC | - | C | A | | |
| G1602a4 | WI-18262 | HT1903 | 1346 | proto-oncogene pim-1, ? | AGCAGCCTTTCTGGC[A/T]GGTCCTCCCCTCTCT | - | A | T | | |
| G1611a1 | WI-18257 | HT1316 | 1199 | RB1, retinoblastoma 1 | CAGTTTTGAAACACA[G/C]AGAACACCACGAAAA | M | G | C | Q | H |
| | | | | (including osteosarcoma) | | | | | | |
| G1623a1 | WI-18469 | HT2205 | 350 | TP53, tumor protein p53 (Li- | CAGAGGCTGCTCCCC[G/C]CGTGGCCCCTGCACC | M | G | C | R | P |
| | | | | Fraumeni syndrome) | | | | | | |
| G1630a6 | WI-18255 | HT3563 | 951 | DCC, deleted in colorectal | CACATATAAAAATGA[G/A]AATATTAGTGCCTCT | S | G | A | E | E |
| | | | | carcinoma | | | | | | |
| G1630a7 | WI-18256 | HT3563 | 1995 | DCC, deleted in colorectal | TCGACACAGAAAGAC[G/A]ACCCGCAGGGGTGAG | S | G | A | T | T |
| | | | | carcinoma | | | | | | |
| G1632a4 | WI-18666 | HT27355 | 680 | tumor suppressor, PDGF | TCGGCCAAAGTCACG[C/T]TCCACAGGGAATTCC | M | C | T | L | F |
| | | | | receptor beta-like, ? | | | | | | |
| G1632a5 | WI-18667 | HT27355 | 853 | tumor suppressor, PDGF | GTACCAGCTGCTCTA[C/T]GTGGCGGTTCCCAGT | S | C | T | Y | Y |
| | | | | receptor beta-like, ? | | | | | | |
| G1633a8 | WI-18663 | HT1778 | 2619 | FER, fer (fps/fes related) | GAGTGACGTGTGGAG[C/T]TTTGGCATCCTTCTC | S | C | T | S | S |
| | | | | tyrosine kinase | | | | | | |
| | | | | (phosphoprotein NCP94) | | | | | | |
| G1633a9 | WI-18668 | HT1778 | 2136 | FER, fer (fps/fes related) | GGGCACATTAAAGGA[T/C]AAAACTTCTGTTGCT | S | T | C | D | D |
| | | | | tyrosine kinase | | | | | | |
| | | | | (phosphoprotein NCP94) | | | | | | |
| G1635a1 | WI-19559 | HT1472 | 1353 | LCK, lymphocyte-specific | GCTGACGGAAATTGT[C/A]ACCCACGGCCGCATC | S | C | A | V | V |
| | | | | protein tyrosine kinase | | | | | | |
| G1645a9 | WI-18664 | D21089 | 247 | XPC, xeroderma pigmentosum, | CCAAAGAAGAGCCTT[C/T]TCTCCAAAGTTTCAC | M | C | T | L | F |
| | | | | complementation group C | | | | | | |
| G1645a10 | WI-18665 | D21089 | 3024 | XPC, xeroderma pigmentosum, | CCCTGGTGGTGGGGG[G/C]TTCTCTGCTGAGAAG | - | G | C | | |
| | | | | complementation group C | | | | | | |
| G1645a11 | WI-18669 | D21089 | 1636 | XPC, xeroderma pigmentosum, | AGCAGTAAAAGAGGC[A/C]AGAAAATGTGCAGCG | M | A | C | K | Q |
| | | | | complementation group C | | | | | | |
| G167a13 | WI-18655 | HT4579 | 1890 | PMS2L8, postmeiotic | CCTGGACTTTTCTAT[G/A]AGTTCTTTAGCTAAA | M | G | A | M | I |
| | | | | segregation increased 2-like | | | | | | |
| | | | | 8 | | | | | | |
| G185a13 | WI-19560 | X77533 | 183 | ACVR2B, activin A receptor, | AGCGGCTGCACTGCT[A/G]CGCCTCCTGGGCCAA | M | A | G | Y | C |
| | | | | type IIB | | | | | | |
| G185a14 | WI-19561 | X77533 | 272 | ACVR2B, activin A receptor, | TACGATAGGCAGGAG[T/G]GTGTGGCCACTGAGG | M | T | G | C | G |
| | | | | type IIB | | | | | | |
| G188a1 | WI-18440 | AB000221 | 234 | SYCA3, small inducible | CCAGTGCCCCAAGCC[A/G]GGTGTCATCCTCCTA | S | A | G | P | P |
| | | | | cytokine A3 (homologous to | | | | | | |
| | | | | mouse Mip-1a) | | | | | | |
| G188a2 | WI-18441 | AB000221 | 289 | SCYA3, small inducible | GCTGACCCCAATAAG[A/T]AGTGGGTCCAGAAAT | N | A | T | K | * |
| | | | | cytokine A3 (homologous to | | | | | | |
| | | | | mouse Mip-1a) | | | | | | |
| G192a1 | WI-18481 | D12614 | 292 | LTA, lymphotoxin alpha (TNF | AGACTGCCCGTCAGC[A/C]CCCCAAGATGCATCT | M | A | C | H | P |
| | | | | superfamily, member 1) | | | | | | |
| G192a2 | WI-18482 | D12614 | 319 | LTA, lymphotoxin alpha (TNF | ATCTTGCCCACAGCA[C/A]CCTCAAACCTGCTGC | M | C | A | T | N |
| | | | | superfamily, member 1) | | | | | | |
| G192a3 | WI-18483 | D12614 | 177 | LTA, lymphotoxin alpha (TNF | TTCCTCCCAAGGGTG[T/C]GTGGCACCACCCTAC | M | T | C | C | R |
| | | | | superfamily, member 1) | | | | | | |
| G197a3 | WI-18206 | D50403 | 1825 | NRAMP1, natural resistance- | TGCAGGCAGCAGGAT[G/A]GAGTGGGACAGTTCC | - | G | A | | |
| | | | | associated macrophage | | | | | | |
| | | | | protein 1 (might include | | | | | | |
| | | | | Leishmaniasis) | | | | | | |
| G197a4 | WI-18962 | D50403 | 737 | NRAMP1, natural resistance- | TGAGTATGTGGTGGC[G/A]CGTCCTGAGCAGGGA | S | G | A | A | A |
| | | | | associated macrophage | | | | | | |
| | | | | protein 1 (might include | | | | | | |
| | | | | Leishmaniasis) | | | | | | |
| G208a2 | WI-18504 | L31581 | 528 | CCR7, chemokine (C-C motif) | CATCAGCATTGACCG[C/A]TACGTGGCCATCGTC | S | C | A | R | R |
| | | | | receptor 7 | | | | | | |
| G208a3 | WI-18678 | L31581 | 975 | CCR7, chemokine (C-C motif) | TGAGCTCAGTAAGCA[A/G]CTCAACATCGCCTAC | S | A | G | Q | Q |
| | | | | receptor 7 | | | | | | |
| G212a1 | WI-19161 | M24854 | 624 | FCGR3A, Fc fragment of IgG, | TTCTGCAGGGGGCTT[G/T]TTGGGAGTAAAAATG | M | G | T | V | F |
| | | | | low affinity IIIa, receptor | | | | | | |
| | | | | for (CD16) | | | | | | |
| G215a7 | WI-18653 | M28393 | 900 | PRF1, perforin 1 | CTACCGGGAGCGCCA[T/C]TCGGAAGTGGTTGGC | S | T | C | H | H |
| | | | | (preforming protein) | | | | | | |
| G215a8 | WI-19278 | M28393 | 1330 | PRF1, perforin 1 | GTGAAGCTCTTCTTT[G/C]GTGGCCAGGAGCTGA | M | G | C | G | R |
| | | | | (preforming protein) | | | | | | |
| G217a7 | WI-18433 | M31932 | 195 | FCGR2B, Fc fragment of IgG, | TGACTCTGACATGCC[A/G]GGGGGCTCGCAGCCC | M | A | G | Q | R |
| | | | | low affinity IIb, receptor | | | | | | |
| | | | | for (CD32) | | | | | | |
| G217a8 | WI-18434 | M31932 | 507 | FCGR2B, Fc fragment of IgG, | CCCAGAAATTCTCCC[G/A]TTTGGATCCCACCTT | M | G | A | R | H |
| | | | | low affinity IIb, receptor | | | | | | |
| | | | | for (CD32) | | | | | | |
| G217a9 | WI-18446 | M31932 | 652 | FCGR2B, Fc fragment of IgG, | GGGCAGCTCTTCACC[A/G]ATGGGGATCATTGTG | S | A | G | P | P |
| | | | | low affinity IIb, receptor | | | | | | |
| | | | | for (CD32) | | | | | | |
| G217a10 | WI-18447 | M31932 | 904 | FCGR2B, Fc fragment of IgG, | GGCACCTACTGACGA[T/C]GATAAAAACATCTAC | S | T | C | D | D |
| | | | | low affinity IIb, receptor | | | | | | |
| | | | | for (CD32) | | | | | | |
| G218a12 | WI-18512 | M36712 | 729 | CD8B1, CD8 antigen, beta | GACATCGGTCAGTAA[T/C]GAGCACGATGTGGAA | - | T | C | | |
| | | | | polypeptide 1 (p37) | | | | | | |
| G218a13 | WI-18513 | M36712 | 820 | CD8B1, CD8 antigen, beta | TTTCACTGCTGCAAG[G/A]CCTTTCTGTGTGTGA | - | G | A | | |
| | | | | polypeptide 1 (p37) | | | | | | |
| G218a14 | WI-18681 | M36712 | 221 | CD8B1, CD8 antigen, beta | GGCTGAGACAGCGCC[A/T]GGCACCGAGCAGTGA | M | A | T | Q | L |
| | | | | polypeptide 1 (p37) | | | | | | |
| G227a3 | WI-18442 | M86511 | 1090 | CD14, CD14 antigen | CCTGGAACTGCCCTC[C/T]CCCACGAGGGCTCAA | M | C | T | P | S |
| G2273a2 | WI-18171 | AF004883 | 4979 | CACNA1A, calcium channel, | GGCCATGATCGCCCT[C/A]AACACCATCGTGCTT | S | C | A | L | L |
| | | | | voltage-dependent, P/Q type, | | | | | | |
| | | | | alpha 1A subunit | | | | | | |
| G228a3 | WI-18435 | U00672 | 241 | IL10RA, interleukin 10 | CTGCTATGAAGTGGC[G/A]CTCCTGAGGTATGGA | S | G | A | A | A |
| | | | | receptor, alpha | | | | | | |
| G228a4 | WI-18436 | U00672 | 1112 | IL10RA, interleukin 10 | AGAACGCTGGGAAAC[G/A]GGGAGCCCCCTGTGC | M | G | A | G | R |
| | | | | receptor, alpha | | | | | | |
| G228a5 | WI-18437 | U00672 | 1320 | IL10RA, interleukin 10 | ACACACAGGGTGGCT[C/T]GGCCTTGGGCCACCA | M | C | T | S | L |
| | | | | receptor, alpha | | | | | | |
| G228a6 | WI-18438 | U00672 | 1033 | IL10RA, interleukin 10 | TGGCTTTGGCAGCAC[C/T]AAGCCATCCCTGCAG | S | C | T | T | T |
| | | | | receptor, alpha | | | | | | |
| G2288a5 | WI-18532 | D29634 | 701 | PTGIR, prostaglandin 12 | CCCTCAGCCTCTGCC[G/A]CATGTACCGCCAGCA | M | G | A | R | H |
| | | | | (prostacyclin) receptor (IP) | | | | | | |
| G2295a5 | WI-18475 | D89079 | 1524 | LTB4R, leukotriene b4 | GAAGAAGAGGGAGAG[A/G]TGGAGCAAAGTGAGG | - | A | G | | |
| | | | | receptor (chemokine receptor- | | | | | | |
| | | | | like 1) | | | | | | |
| G230a2 | WI-18448 | U31628 | 892 | IL15RA, interleukin 15 | CCACCTATGAAACTC[G/A]GGGAAACCAGCCCAG | - | G | A | | |
| | | | | receptor, alpha | | | | | | |
| G231a2 | WI-19159 | U32324 | 189 | IL11RA, interleukin 11 | GGCAGCCAGGGAGGT[C/T]CGTGAAGCTGTGTTG | M | C | T | S | F |
| | | | | receptor, alpha | | | | | | |
| G2314a3 | WI-18265 | J05272 | 1999 | IMPDH1, IMP (inosine | GCAGGCATCCAACAC[G/T]GCTGCCAGGATATCG | M | G | T | G | C |
| | | | | monophosphate) dehydrogenase | | | | | | |
| | | | | 1 | | | | | | |
| G2316a1 | WI-18817 | J05594 | 173 | HPGD, hydroxyprostaglandin | TGCCCTGCATGAGCA[A/G]TTTGAACCTCAGAAG | S | A | G | Q | Q |
| | | | | dehydrogenase 15-(NAD) | | | | | | |
| G2330a6 | WI-18686 | L22607 | 1007 | ADORA3, adenosine A3 | TGGCTGCCTTTATCT[A/C]TCATCAACTGCATCA | M | A | C | I | L |
| | | | | receptor | | | | | | |
| G2330a7 | WI-18687 | L22607 | 1134 | ADORA3, adenosine A3 | AAATAAAGAAGTTCA[A/T]GGAAACCTACCTTTT | M | A | T | K | M |
| | | | | receptor | | | | | | |
| G2335a9 | WI-18266 | L32961 | 492 | ABAT, 4-aminobutyrate | AACAGACCCGCCCTC[G/A]AAATCCTGCCTCCGG | M | G | A | E | K |
| | | | | aminotransferase | | | | | | |
| G2335a10 | WI-18267 | L32961 | 1114 | ABAT, 4-aminobutyrate | GCACGGGCAAGTTCT[G/A]GGCCCATGAGCACTG | N | G | A | W | * |
| | | | | aminotransferase | | | | | | |
| G2335a11 | WI-18268 | L32961 | 1245 | ABAT, 4-aminobutyrate | ATCTTCAACACGTGG[C/T]TGGGGGACCCGTCCA | S | C | T | L | L |
| | | | | aminotransferase | | | | | | |
| G2355a4 | WI-18500 | M16405 | 1963 | CHRM4, cholinergic | AGGTGCGCAAGAAGC[G/A]GCAGATGGCGGCCCG | M | G | A | R | Q |
| | | | | receptor, muscarinic 4 | | | | | | |
| G236a4 | WI-19162 | U84487 | 628 | SCYD1, small inducible | AGGGCCTGTGGGCAC[G/T]GAGCTTTTCCGAGTG | S | G | T | T | T |
| | | | | cytokine subfamily D (Cys-X3- | | | | | | |
| | | | | Cys), member 1 (fractalkine, | | | | | | |
| | | | | neurotactin) | | | | | | |
| G236a5 | WI-19163 | U84487 | 728 | SCYD1, small inducible | GAGGCAAAGACCTCT[G/A]AGGCCCCGTCCACCC | M | G | A | E | K |
| | | | | cytokine subfamily D (Cys-X3- | | | | | | |
| | | | | Cys), member 1 (fractalkine, | | | | | | |
| | | | | neurotactin) | | | | | | |
| G2363a7 | WI-19439 | HT4822 | 673 | CSF1, colony stimulating | TACAGGTGGAGGCGG[C/A]GGAGCCATCAAGAGC | M | C | A | | R |
| | | | | factor 1 (macrophage) | | | | | | |
| G2363a8 | WI-19440 | HT4822 | 698 | CSF1, colony stimulating | AAGAGCCTCAGAGAG[C/T]GGATTCTCCCTTGGA | M | C | T | | R |
| | | | | factor 1 (macrophage) | | | | | | |
| G2373a2 | WI-18812 | M36035 | 546 | BZRP, benzodiazapine | ACAACCATGGCTGGC[A/G]TGGGGGACGGCGGCT | M | A | G | H | R |
| | | | | receptor (peripheral) | | | | | | |
| G2376a2 | WI-18813 | M57414 | 1124 | TACR2, tachykinin receptor | GTGGGGAGGCGGGGC[G/A]TCCCCAGGATGGATC | M | G | A | R | H |
| | | | | 2 | | | | | | |
| G2376a3 | WI-18814 | M57414 | 1128 | TACR2, tachykinin receptor | GGAGGCGGGGCGTCC[C/T]CAGGATGGATCAGGG | S | C | T | P | P |
| | | | | 2 | | | | | | |
| G240a2 | WI-19160 | X04391 | 1125 | Human mRNA for lymphocyte | GCTGTCCCAGTGCCA[C/T]GAACTTTGGGAGAGA | S | C | T | H | H |
| | | | | glycoprotein T1/Leu-1., ? | | | | | | |
| G2403a4 | WI-18522 | M83670 | 272 | CA4, carbonic anhydrase IV | CTTCTTCTCTGGCTA[C/T]GATAAGAAGCAAACG | S | C | T | Y | Y |
| G2403a5 | WI-18523 | M83670 | 1003 | CA4, carbonic anhydrase IV | GGCTCACTTCTGCAC[G/A]CAGCCTCTCTGTTGC | - | G | A | | |
| G2409a2 | WI-19006 | M93394 | 745 | AGTR1, angiotensin receptor | CCAAAATTCAACCCT[T/C]CCGATAGGGCTGGGC | S | T | C | L | L |
| | | | | 1 | | | | | | |
| G2425a1 | WI-19567 | U03865 | 294 | ADRA1B, adrenergic, alpha- | GGGCGCCTTCATCCT[C/T]TTTGCCATCGTGGGC | S | C | T | L | L |
| | | | | 1B-, receptor | | | | | | |
| G2425a2 | WI-19568 | U03865 | 417 | ADRA1B, adrenergic, alpha- | GTTGAGCTTCACCGT[C/T]CTGCCCTTCTCAGCG | S | C | T | V | V |
| | | | | 1B-, receptor | | | | | | |
| G2425a3 | WI-19569 | U03865 | 502 | ADRA1B, adrenergic, alpha- | GCAGCCGTGGATGTC[C/T]TGTGCTGCACAGCGT | S | C | T | L | L |
| | | | | 1B-, receptor | | | | | | |
| G2425a4 | WI-19570 | U03865 | 672 | ADRA1B, adrenergic, alpha- | CGGGCCTCTCCTTGG[G/A]TGGAAGGAGCCGGCA | S | G | A | G | G |
| | | | | 1B-, receptor | | | | | | |
| G2430a1 | WI-19571 | U09353 | 520 | LCT4S, leukotriene C4 | TGAGACCAAGGCCCC[C/T]GGGCCGACGGAGCCG | - | C | T | | |
| | | | | synthase | | | | | | |
| G2452a1 | WI-18235 | U63970 | 3127 | CMOAT, canalicular | AGTCTACGGAGCTCT[G/A]GGATTAGCCCAAGGT | S | G | A | L | L |
| | | | | multispecific organic anion | | | | | | |
| | | | | transporter | | | | | | |
| G2452a2 | WI-18392 | U63970 | 2583 | CMOAT, canalicular | CCTACAGTGCTCTCC[T/G]GGCCAAAAAAGGAGA | M | T | G | L | R |
| | | | | multispecific organic anion | | | | | | |
| | | | | transporter | | | | | | |
| G2452a3 | WI-18393 | U63970 | 4327 | CMOAT, canalicular | GTTATCCCACGAAGT[G/T]ACAGAGGCTGGTGGC | S | G | T | V | V |
| | | | | multispecific organic anion | | | | | | |
| | | | | transporter | | | | | | |
| G2482a1 | WI-19167 | X56088 | 1004 | CYP7A1, cytochrome P450, | GAACATTAGAGAATG[C/T]TGGTCAAAAAGTCAG | M | C | T | A | V |
| | | | | subfamily VIIA (chloesterol | | | | | | |
| | | | | 7 alpha-monooxygenase), | | | | | | |
| | | | | polypeptide 1 | | | | | | |
| G250a3 | WI-19279 | HT0155 | 521 | IL3RA, interleukin 3 | GAGCTGCAGCTGGGC[G/A]GTAGGCCCGGGGGCC | S | G | A | A | A |
| | | | | receptor, alpha (low | | | | | | |
| | | | | affinity) | | | | | | |
| G2513a19 | WI-18372 | HT27365 | 1242 | PLCB3, phospholipase C, | CGAAGCGTTGAACTC[G/A]ATGTAAGTGATGGTT | M | G | A | D | N |
| | | | | beta 3 (phosphatidylinositol- | | | | | | |
| | | | | specific) | | | | | | |
| G2513a20 | WI-18373 | HT27365 | 1269 | PLCB3, phospholipase C, | GGTTCAGATAATGAA[C/T]CAATCCTTTGTAATC | M | C | T | P | S |
| | | | | beta 3 (phosphatidylinositol- | | | | | | |
| | | | | specific) | | | | | | |
| G2513a21 | WI-18374 | HT27365 | 1616 | PLCB3, phospholipase C, | GTCTCGAAGGATGTC[G/A]GTAGATTACAATGGT | S | G | A | S | S |
| | | | | beta 3 (phosphatidylinositol- | | | | | | |
| | | | | specific) | | | | | | |
| G2513a22 | WI-18375 | HT27365 | 2399 | PLCB3, phospholipase C, | TGTTCCCCTGCGTTC[T/C]TTTGTGGGTGACATC | S | T | C | S | S |
| | | | | beta 3, (phosphatidylinositol- | | | | | | |
| | | | | specific) | | | | | | |
| G2513a23 | WI-18376 | HT27365 | 2430 | PLCB3, phospholipase C, | ATGGAGCACGTAACC[C/T]TTTTTGTCCACATAG | M | C | T | L | F |
| | | | | beta 3 (phosphatidylinositol- | | | | | | |
| | | | | specific) | | | | | | |
| G2513a24 | WI-18377 | HT27365 | 2756 | PLCB3, phospholipase C, | ACTTGTGATGAAAGA[C/T]AGCTTTCCTTACCTG | S | C | T | D | D |
| | | | | beta 3 (phosphatidylinositol- | | | | | | |
| | | | | specific) | | | | | | |
| G2513a25 | WI-18378 | HT27365 | 3006 | PLCB3, phospholipase C, | GCTTGGAACATTACA[G/A]TATTGAAGGGCCAAG | M | G | A | V | I |
| | | | | beta 3 (phosphatidylinositol- | | | | | | |
| | | | | specific) | | | | | | |
| G2513a26 | WI-18379 | HT27365 | 3137 | PLCB3, phospholipase C, | TGCTGAGGCCAAGAG[C/T]AAGCGCAGCCTGGAA | S | C | T | S | S |
| | | | | beta 3 (phosphatidtlinositol- | | | | | | |
| | | | | specific) | | | | | | |
| G2514a2 | WI-19572 | Z46632 | 1142 | PDE4C, phosphodiesterase | ACGTAAGTGGGAACC[G/A]GCCCCTCACAGCTAT | M | G | A | R | Q |
| | | | | 4C, cAMP-specific (dunce | | | | | | |
| | | | | (Drosophila)-homolog | | | | | | |
| | | | | phosphodiesterase E1) | | | | | | |
| G2514a3 | WI-19573 | Z46632 | 1245 | PDE4C, phosphodiesterase | CCTGCTGATGCTGGA[G/A]GGTCACTACCACGCC | S | G | A | E | E |
| | | | | 4C, cAMP-specific (dunce | | | | | | |
| | | | | (Drosophila)-homolog | | | | | | |
| | | | | phosphodiesterase E1) | | | | | | |
| G2514a4 | WI-19574 | Z46632 | 1259 | PDE4C, phosphodiesterase | AGGGTCACTACCACG[C/T]CAATGTGGCCTACCA | M | C | T | A | V |
| | | | | 4C, cAMP-specific (dunce | | | | | | |
| | | | | (Drosophila)-homolog | | | | | | |
| | | | | phosphodiesterase E1) | | | | | | |
| G2514a5 | WI-19575 | Z46632 | 1269 | PDE4C, phosphodiesterase | CCACGCCAATGTGGC[C/T]TACCACAACAGCCTA | S | C | T | A | A |
| | | | | 4C, cAMP-specific (dunce | | | | | | |
| | | | | (Drosophila)-homolog | | | | | | |
| | | | | phosphodiesterase E1) | | | | | | |
| G2514a6 | WI-19576 | Z46632 | 1332 | PDE4C, phosphodiesterase | GCTGCTGGCTACGCC[C/T]GCCCTCGAGGCTGTG | S | C | T | P | P |
| | | | | 4C, cAMP-specific (dunce | | | | | | |
| | | | | (Drosophila)-homolog | | | | | | |
| | | | | phosphodiesterase E1) | | | | | | |
| G252a3 | WI-18449 | HT0425 | 912 | FCER2, Fc fragment of IgE, | GAAGGGAGAGTTTAT[C/T]TGGGTGGATGGGAGC | S | C | T | I | I |
| | | | | low affinity II, receptor | | | | | | |
| | | | | for (CD23A) | | | | | | |
| G252a4 | WI-18450 | HT0425 | 1032 | FCER2, Fc fragment of IgE, | GAACGACGCCTTCTG[C/T]GACCGTAAGCTGGGC | S | C | T | C | C |
| | | | | low affinity II, receptor | | | | | | |
| | | | | for (CD23A) | | | | | | |
| G253a2 | WI-18484 | HT0573 | 303 | IFNB1, interferon, beta 1, | CCAGAAGGAGGACGC[C/T]GCATTGACCATCTAT | S | C | T | A | A |
| | | | | fibroblast | | | | | | |
| G253a3 | WI-18526 | HT0573 | 537 | IFNB1, interferon, beta 1, | GATTCTGCATTACCT[G/A]AAGGCCAAGGAGTAC | S | G | A | L | L |
| | | | | fibroblast | | | | | | |
| G254a4 | WI-18451 | HT0611 | 987 | IL4R, interleukin 4 | TTCCCAACCCAGCCC[G/A]CAGCCGCCTCGTGGC | M | G | A | R | H |
| | | | | receptor | | | | | | |
| G254a5 | WI-18452 | HT0611 | 1682 | IL4R, interleukin 4 | CGCAGCTTCAGCAAC[T/C]CCCTGAGCCAGTCAC | M | T | C | S | P |
| | | | | receptor | | | | | | |
| G261a3 | WI-18439 | HT1101 | 349 | IL7R, interleukin 7 | GAGCAATATATGTGT[G/A]AAGGTTGGAGAAAAG | S | G | A | V | V |
| | | | | receptor | | | | | | |
| G261a4 | WI-18443 | HT1101 | 1260 | IL7R, interleukin 7 | TTGGGACTACAAACA[G/A]CACGCTGCCCCCTCC | M | G | A | S | N |
| | | | | receptor | | | | | | |
| G261a5 | WI-18444 | HT1101 | 1263 | IL7R, interleukin 7 | GGACTACAAACAGCA[C/T]GCTGCCCCCTCCATT | M | C | T | T | M |
| | | | | receptor | | | | | | |
| G261a6 | WI-18445 | HT1101 | 1366 | IL7R, interleukin 7 | AAATCAAGAAGAAGC[A/T]TATGTCACCATGTCC | S | A | T | A | A |
| | | | | receptor | | | | | | |
| G261a7 | WI-18453 | HT1101 | 753 | IL7R, interleukin 7 | ATCCTATCTTACTAA[C/T]CATCAGCATTTTGAG | M | C | T | T | I |
| | | | | receptor | | | | | | |
| G261a8 | WI-18454 | HT1101 | 1088 | IL7R, interleukin 7 | TCTGAGGATGTAGTC[G/A]TCACTCCAGAAAGCT | M | G | A | V | I |
| | | | | receptor | | | | | | |
| G2648a1 | WI-18240 | HT2947 | 946 | HSD17B1, hydroxysteroid (17- | GACGAGGCCGGGCGC[A/G]GTGCGGTGGGGGACC | M | A | G | S | G |
| | | | | beta) dehydrogenase 1 | | | | | | |
| G2648a2 | WI-18241 | HT2947 | 1070 | HSD17B1, hydroxysteroid (17- | CTGGGGATGGGGCGG[C/T]GGTAGCAGCTGTGGG | - | C | T | | |
| | | | | beta) dehydrogenase 1 | | | | | | |
| G266a2 | WI-19349 | M55646 | 458 | IL1RN, interleukin 1 | CATCCGCTCAGACAG[T/C]GGCCCCACCACCAGT | S | T | C | S | S |
| | | | | receptor antagonist | | | | | | |
| G266a3 | WI-19350 | M55646 | 471 | IL1RN, interleukin 1 | AGTGGCCCCACCACC[A/G]GTTTTGAGTCTGCCG | M | A | G | S | G |
| | | | | receptor antagonist | | | | | | |
| G266a4 | WI-19351 | M55646 | 239 | IL1RN, interleukin 1 | CAACCAACTAGTTGC[T/C]GGATACTTGCAAGGA | S | T | C | A | A |
| | | | | receptor antagonist | | | | | | |
| G27a1 | WI-18242 | M73832 | 1004 | CSF2RA, colony stimulating | CCCGCCAGTTCCACA[G/C]ATCAAAGACAAACTG | M | G | C | | D |
| | | | | factor 2 receptor, alpha, | | | | | | |
| | | | | low-affinity (granulocyte- | | | | | | |
| | | | | macrophage) | | | | | | |
| G27a2 | WI-18243 | M73832 | 1133 | CSF2RA, colony stimulating | TACCTGAGACCCAGA[G/A]GGTGTAGGAATGGCA | - | G | A | | |
| | | | | factor 2 receptor, alpha, | | | | | | |
| | | | | low-affinity (granulocyte- | | | | | | |
| | | | | macrophage) | | | | | | |
| G275a1 | WI-18961 | HT3654 | 1048 | CD8A, CD8 antigen, alpha | ACGACGCCAGCGCCG[C/A]GACCACCAACACCGG | S | C | A | R | R |
| | | | | polypeptide (p32) | | | | | | |
| G275a2 | WI-19164 | HT3654 | 810 | CD8A, CD8 antigen, alpha | GCCGCGCGGCGCCGC[C/A]GCCAGTCCCACCTTC | S | C | A | A | A |
| | | | | polypeptide (p32) | | | | | | |
| G275a3 | WI-19165 | HT3654 | 897 | CD8A, CD8 antigen, alpha | GTTCTCGGGCAAGAG[G/A]TTGGGGGACACCTTC | S | G | A | R | R |
| | | | | polypeptide (p32) | | | | | | |
| G276a4 | WI-18505 | HT3670 | 681 | CD4 antigen, ? | CCAAGGGGTAAAAAC[A/C]TACAGGGGGGGAAGA | M | A | C | I | L |
| G276a5 | WI-18506 | HT3670 | 874 | CD4 antigen, ? | CCTTCCCACTCGCCT[T/C]TACAGTTGAAAAGCT | M | T | C | F | S |
| G276a6 | WI-18507 | HT3670 | 893 | CD4 antigen, ? | AGTTGAAAAGCTGAC[G/T]GGCAGTGGCGAGCTG | S | G | T | T | T |
| G276a7 | WI-18508 | HT3670 | 987 | CD4 antigen, ? | GAAGTGTCTGTAAAA[C/T]GGGTTACCCAGGACC | M | C | T | R | W |
| G276a8 | WI-18509 | HT3670 | 1486 | CD4 antigen, ? | GGCGCCAAGCAGAGC[G/A]GATGTCTCAGATCAA | M | G | A | R | Q |
| G276a9 | WI-18510 | HT3670 | 1645 | CD4 antigen, ? | TGCCTGCGGACCAGA[T/A]GAATGTAGCAGATCC | - | T | A | | |
| G276a10 | WI-18511 | HT3670 | 1668 | CD4 antigen, ? | GCAGATCCCCAGCCT[C/T]TGGCCTCCTGTTCGC | - | C | T | | |
| G276a11 | WI-18679 | HT3670 | 1079 | CD4 antigen, ? | GTATGCTGGCTCTGG[A/G]AACCTCACCCTGGCC | S | A | G | G | G |
| G276a12 | WI-18680 | HT3670 | 1201 | CD4 antigen, ? | GGGGACCCACCTCCC[C/T]TAAGCTGATGCTGAG | M | C | T | P | L |
| G279a21 | WI-18791 | K01740 | 1500 | F8C, coagulation factor | CCGATTTATGGCATA[C/T]ACAGATGAAACCTTT | S | C | T | Y | Y |
| | | | | VIIIc, procoagulant | | | | | | |
| | | | | component (hemophilia A) | | | | | | |
| G281a5 | WI-18797 | L06105 | 701 | FDFT1, farnesyl-diphosphate | AAACATCATCCGTGA[C/T]TATCTGGAAGACCAG | S | C | T | D | D |
| | | | | farnesyltransferase 1 | | | | | | |
| G2959a3 | WI-18174 | HT0134 | 1624 | GRLF1, glucocorticoid | GCTCAAGAAATTGAC[G/A]GAAGGTTCACAAGCA | M | G | A | G | R |
| | | | | receptor DNA binding factor | | | | | | |
| | | | | 1 | | | | | | |
| G297a6 | WI-18796 | U16660 | 738 | ECH1, enoyl Coenzyme A | GATGGCTGACGAGGC[C/T]CTGGACAGTGGGCTG | S | C | T | A | A |
| | | | | hydratase 1, peroxisomal | | | | | | |
| G2982a2 | WI-18533 | HT0358 | 1802 | homeotic protein 7, notch | GTCACAGTGGTCACC[T/A]CCAGGGTGAGCATCC | M | T | A | L | H |
| | | | | group, ? | | | | | | |
| G2991a1 | WI-18476 | HT0534 | 221 | ZFP36, zinc finger protein | GTCACCTCCCGCCTG[C/T]CTGGCCGCTCCACCA | M | C | T | P | S |
| | | | | homologous to zfp-36 in | | | | | | |
| | | | | mouse | | | | | | |
| G3012a3 | WI-18264 | HT0873 | 761 | MAD, MAX dimerization | GCTATTCCAGCACCA[G/A]CATCAAGAGAATAAA | M | G | A | S | N |
| | | | | protein | | | | | | |
| G3023a9 | WI-18170 | HT0966 | 366 | zinc finger, X-linked, | GTTTTCCTGCTCTTT[C/T]CCTGGCTGCAGCAAG | S | C | T | F | F |
| | | | | duplicated A, ? | | | | | | |
| G3029a4 | WI-18536 | HT1100 | 813 | zinc finger protein 8, ? | CTCCCTCGTCCAGCA[T/C]GAGCGCATCCACACT | S | T | C | H | H |
| G3029a5 | WI-18537 | HT1100 | 1703 | zinc finger protein 8, ? | ATAGTGTACTCATGG[A/G]AGGAGGGGCTGGGGG | - | A | G | | |
| G3034a3 | WI-18496 | HT1182 | 178 | TCF12, transcription factor | AGCTTGGCTTTATCA[A/G]CCAGAGACCGAGGCT | M | A | G | T | A |
| | | | | 12 (HTF4, helix-loop-helix | | | | | | |
| | | | | transcription factors 4) | | | | | | |
| G3050a8 | WI-18806 | HT1558 | 1748 | ?, ? | ATACCTACCACTGTC[C/T]TCAACATTCCCCACC | M | C | T | L | F |
| G3050a9 | WI-18807 | HT1558 | 2704 | ?, ? | AAAAGAGAAAAAGAA[G/T]AAACGGAAGGCAGAG | M | G | T | K | N |
| G3050a10 | WI-18808 | HT1558 | 3178 | ?, ? | GAAACCCCGGAAGCC[C/T]TACACCATTAAGAAG | S | C | T | P | P |
| G3057a23 | WI-18688 | HT1669 | 7874 | alpha-fetoprotein enhancer- | AGATGGTGCTTCACG[T/C]CCCCACCGGCGGCGG | M | T | C | V | A |
| | | | | binding protein, ? | | | | | | |
| G3114a2 | WI-19564 | HT2617 | 588 | GTF2E2, general | CCAAAATTGAAGTAA[T/C]AGATGGGAAGTATGC | M | T | C | I | T |
| | | | | transcription factor IIE, | | | | | | |
| | | | | polypeptide 2 (beta subunit, | | | | | | |
| | | | | 34kD) | | | | | | |
| G3118a3 | WI-18514 | HT2652 | 1241 | ZNF35, zinc finger protein | GCTCAAACCTCATTG[T/C]CCACCAGAGGATCCA | M | T | C | V | A |
| | | | | 35 (clone HF.10) | | | | | | |
| G3119a8 | WI-18515 | HT2654 | 2179 | GLI, glioma-associated | GCTGCCATGGATGCT[A/G]GAGGGCTACAGGAAG | M | A | G | R | G |
| | | | | oncogene homolog (zinc | | | | | | |
| | | | | finger protein) | | | | | | |
| G3119a9 | WI-18516 | HT2654 | 2300 | GLI, glioma-associated | AAGGGGCAGCAGCTG[A/G]GCCTTATGGAGCGAG | M | A | G | E | G |
| | | | | oncogene homolog (zinc | | | | | | |
| | | | | finger protein) | | | | | | |
| G3119a10 | WI-18517 | HT2654 | 3113 | GLI, glioma-associated | CAAACCCCAGCTGTG[G/T]TCATCCTGAGGTGGG | M | G | T | G | V |
| | | | | oncogene homolog (zinc | | | | | | |
| | | | | finger protein) | | | | | | |
| G3122a1 | WI-18518 | HT2671 | 1177 | HOXB2, homeo box B2 | TCCCGGTCCTTTCGA[C/T]CCCCGCGCTCCTTGG | - | C | T | | |
| G3124a2 | WI-18525 | HT2673 | 607 | HOXB3, homeo box B3 | GGTCTGGCCCCCGAG[A/C]CCCTGTCGGCCCCGC | M | A | C | T | P |
| G3129a2 | WI-18527 | HT2695 | 1138 | transcription factor ATF-a, | GCGCAACCGGGCTGC[A/G]GCCTCCCGCTGCCGC | S | A | G | A | A |
| | | | | ? | | | | | | |
| G313a9 | WI-19158 | HT0462 | 1948 | platelet-derived growth | CTTGGGGTCTGGAGC[G/A]TTTGGGAAGGTGGTT | S | G | A | A | A |
| | | | | factor, alpha polypeptide | | | | | | |
| | | | | (GB:M21574), ? | | | | | | |
| G3168a1 | WI-18528 | HT27665 | 1364 | zinc-finger protein | CATTGATAGCTTTGT[G/A]CTAAGCTTCCTTGGG | S | G | A | V | V |
| | | | | (GB:U18543), ? | | | | | | |
| G3173a4 | WI-18529 | HT2772 | 729 | ZNF74, zinc finger protein | GTTCCGCCAGAGCTC[C/T]TCCCTCACGCTGCAC | S | C | T | S | S |
| | | | | 74 (Cos52) | | | | | | |
| G3175a2 | WI-18530 | HT2776 | 3882 | transcriptional regulator, | CATCTTGGAGCATGA[A/G]GAGGAAAATGAGGAA | S | A | G | E | E |
| | | | | via glucocorticoid receptor, | | | | | | |
| | | | | ? | | | | | | |
| G3175a3 | WI-18531 | HT2776 | 4053 | transcriptional regulator, | GGAGGATGAGCTGCC[C/T]TCCTGGATCATTAAG | S | C | T | P | P |
| | | | | via glucocorticoid receptor, | | | | | | |
| | | | | ? | | | | | | |
| G303a2 | WI-18283 | HT3523 | 1012 | POU6F1, POU domain, class | AAGGAGCTCAACTAC[G/A]ACCGTGAGGTAGTGC | M | G | A | D | N |
| | | | | 6, transcription factor 1 | | | | | | |
| G3305a1 | WI-18295 | HT3549 | 791 | AES, amino-terminal | CCAGCCCAGCTTGCA[G/A]GCCACCTCTAGCTTT | - | G | A | | |
| | | | | enhancer of split | | | | | | |
| G3320a5 | WI-18284 | HT3622 | 471 | BCL6, B-cell CLL/lymphoma 6 | TAGAGCCCATAAAAC[G/A]GTCCTCATGGCCTGC | S | G | A | T | T |
| | | | | (zinc finger protein 51) | | | | | | |
| G3220a6 | WI-18285 | HT3622 | 690 | BCL6,B-cell CLL/lymphoma 6 | TGTTGTGGACACTTG[C/T]CGGAAGTTTATTAAG | S | C | T | C | C |
| | | | | (zinc finger protein 51) | | | | | | |
| G3358a7 | WI-18286 | HT4187 | 233 | ETV5, ets variant gene 5 | AAGTCCCTTTTATGG[T/C]CCCAGGGAAATCTCG | M | T | C | V | A |
| | | | | (ets-related molecule) | | | | | | |
| G3358a8 | WI-18287 | HT4187 | 467 | ETV5, ets variant gene 5 | CCAAGATCAAACGGG[A/G]GCTGCACAGCCCCTC | M | A | G | E | G |
| | | | | (ets-related molecule) | | | | | | |
| G3396a11 | WI-18288 | HT4491 | 1538 | ZNF135, zinc finger protein | AGCCATAGCTCATCC[C/T]TTTCTAGATTTGACC | - | C | T | | |
| | | | | 135 (clone pHZ-17) | | | | | | |
| G3396a12 | WI-18289 | HT4491 | 1553 | ZNF135, zinc finger protein | CTTTCTAGATTTGAC[C/T]CAATCATACACATGA | - | C | T | | |
| | | | | 135 (clone pHZ-17) | | | | | | |
| G3396a13 | WI-18290 | HT4491 | 1577 | ZNF135, zinc finger protein | CACATGAGAAACGTA[C/A]ATTCATACACAAGCC | - | C | A | | |
| | | | | 135 (clone pHZ-17) | | | | | | |
| G3396a14 | WI-18291 | HT4491 | 1578 | ZNF135, zinc finger protein | ACATGAGAAACGTAC[A/G]TTCATACACAAGCCT | - | A | G | | |
| | | | | 135 (clone pHZ-17) | | | | | | |
| G3396a15 | WI-18292 | HT4491 | 1582 | ZNF135, zinc finger protein | GAGAAACGTACATTC[A/C]TACACAAGCCTTTTC | - | A | C | | |
| | | | | 135 (clone pHZ-17) | | | | | | |
| G3396a16 | WI-18293 | HT4491 | 1587 | ZNF135, zinc finger protein | ACGTACATTCATACA[C/A]AAGCCTTTTCACACA | - | C | A | | |
| | | | | 135 (clone pHZ-17) | | | | | | |
| G3405a3 | WI-18294 | HT4519 | 378 | ILF3, interleukin enhancer | CATGGTGTCCCACAC[G/A]GAGCGGGCGCTCAAA | S | G | A | T | T |
| | | | | binding factor 3, 90kD | | | | | | |
| G345a6 | WI-18402 | HT1729 | 889 | MSR1, macrophage scavenger | TTAATTCAAGGTCCT[C/G]CTGGACCCCCGGGTG | M | C | G | P | A |
| | | | | receptor 1 | | | | | | |
| G371a6 | WI-18272 | HT27943 | 1124 | CRAT, carnitine | ATGGTGCCCCTGCCC[A/C]TGCCCAAGAAGCTGC | M | A | C | M | L |
| | | | | acetyltransferase | | | | | | |
| G391a35 | WI-19166 | HT3630 | 5563 | VWF, von Willebrand factor | CCCCAGCCAAATCGG[G/T]GATGCCTTGGGCTTT | S | G | T | G | G |
| G3941a8 | WI-18172 | HT3464 | 749 | mannosidase, alpha, | TGGAGCAGGTGTGGC[G/A]GGCCAGCACCAGCCT | M | G | A | R | Q |
| | | | | lysosomal, ? | | | | | | |
| G3941a9 | WI-18173 | HT3464 | 777 | mannosidase, alpha, | CCTGAAGCCCCCGAC[C/T]GCGGACCTCTTCACT | S | C | T | T | T |
| | | | | lysosomal, ? | | | | | | |
| G3956a1 | WI-18296 | HT1347 | 895 | KRT18, keratin 18 | GAGAGCACCACAGTG[G/A]TCACCACACAGTCTG | M | G | A | V | I |
| G3959a4 | WI-18297 | HT4490 | 2914 | ADTB1, adaptin, beta 1 | CCCCGGCCAGCGCCC[A/G]CCCCAGCCTTCTGCC | - | A | G | | |
| | | | | (beta prime) | | | | | | |
| G3971a1 | WI-18300 | HT27832 | 152 | CRYBB1, crystallin, beta B1 | AGGGGAAGGGGGCCC[C/T]ACCTGCAGGAACATC | M | C | T | P | L |
| G3971a2 | WI-18301 | HT27832 | 227 | CRYBB1, crystallin, beta B1 | CCAGCGCCAAGGCGG[C/A]GGAACTGCCTCCTGG | M | C | A | A | E |
| G3971a3 | WI-18302 | HT27832 | 331 | CRYBB1, crystallin, beta B1 | AATCTGGCAGACCGT[G/A]GCTTCGACCGTGTGC | M | G | A | G | S |
| G3986a2 | WI-18318 | HT0708 | 580 | TNNC1, troponin C, slow | GTCCTGGGGTTGGGG[A/G]GGGGGTCGGGGTCCC | - | A | G | | |
| G3986a3 | WI-18319 | HT0708 | 582 | TNNC1, troponin C, slow | CCTGGGGTTGGGGAG[G/A]GGGTCGGGGTCCCAG | - | G | A | | |
| G4022a7 | WI-18331 | HT2426 | 812 | TGM1, transglutaminase 1 (K | TGACCCCCGCAATGA[G/A]ATCTACATCCTCTTC | S | G | A | E | E |
| | | | | polypeptide epidermal type | | | | | | |
| | | | | I, protein-glutamine-gamma- | | | | | | |
| | | | | glutamyltransferase) | | | | | | |
| G4022a8 | WI-18332 | HT2426 | 1645 | TGM1, transglutaminase 1 (K | TTGTTTATGTGGAGG[A/G]GAAGGCCATCGGCAC | M | A | G | E | G |
| | | | | polypeptide epidermal type | | | | | | |
| | | | | I, protein-glutamine-gamma- | | | | | | |
| | | | | glutamyltransferase) | | | | | | |
| G4022a9 | WI-18333 | HT2426 | 1905 | TGM1, transglutaminase 1 (K | AATCACAGCAGCAGC[C/T]GCCGCACAGTGAAAC | M | C | T | R | C |
| | | | | polypeptide epidermal type | | | | | | |
| | | | | I, protein-glutamine-gamma- | | | | | | |
| | | | | glutamyltransferase) | | | | | | |
| G4022a10 | WI-18974 | HT2426 | 1232 | TGM1, transglutaminase 1 (K | CTGGGTCTTTGCTGG[C/A]GTGACCACCACAGTG | S | C | A | G | G |
| | | | | polypeptide epidermal type | | | | | | |
| | | | | I, protein-glutamine-gamma- | | | | | | |
| | | | | glutamyltransferase) | | | | | | |
| G4038a18 | WI-18350 | HT4211 | 1275 | LAMB3, laminin, beta 3 | TGATCCGGATGGGGC[A/G]GTCGCAGGGGCTCCC | S | A | G | A | A |
| | | | | (nicein (125kD), kalinin | | | | | | |
| | | | | (140kD), BM600 (125kD)) | | | | | | |
| G4038a19 | WI-18351 | HT4211 | 1429 | LAMB3, laminin, beta 3 | TGCAACATCCTGGGG[T/A]CCCGGGAGATGCCGT | M | T | A | S | T |
| | | | | nicein (125kD), kalinin | | | | | | |
| | | | | (140kD), BM600 (125kD)) | | | | | | |
| G4038a20 | WI-18352 | HT4211 | 1820 | LAMB3, laminin, beta 3 | GTGACCAGTGCCAGC[G/A]AGGCTACTGCAATCG | M | G | A | R | Q |
| | | | | (nicein (125kD), kalinin | | | | | | |
| | | | | (140kD), BM600 (125kD)) | | | | | | |
| G4042a1 | WI-18992 | HT1460 | 448 | TNNC2, troponin C2, fast | CGGGGAGCACGTGAC[T/G]GACGAGGAGATCGAA | S | T | G | T | T |
| G4045a3 | WI-18353 | HT0652 | 1086 | adducin, beta subunit, ? | TCGGATCAACCTGCA[G/A]AAGTGCCTTGGACCC | S | G | A | Q | Q |
| G4080a43 | WI-18199 | HT1396 | 2054 | HSPG2, heparan sulfate | ACCTGGTGCTCTGAA[C/T]CAGCGCCAGGTCCAG | S | C | T | N | N |
| | | | | proteoglycan 2 (perlecan) | | | | | | |
| G4080a44 | WI-18200 | HT1396 | 13032 | HSPG2, heparan sulfate | GAGCTGGTCAGCGGC[C/A]GGTCCCCAGGTCCCA | S | C | A | R | R |
| | | | | proteoglycan 2 (perlecan) | | | | | | |
| G4102a1 | WI-18565 | HT2458 | 1449 | COL5A2, collagen, type V, | CCCAACGGGCTCTCC[G/A]GGTACCTCTGGTCCT | S | G | A | P | P |
| | | | | alpha 2 | | | | | | |
| G4102a2 | WI-18566 | HT2458 | 2039 | COL5A2, collagen, type V, | CAGGAAATCCTGGAG[T/C]TCCTGGGCAAAGGGG | M | T | C | V | A |
| | | | | alpha 2 | | | | | | |
| G4106a2 | WI-18355 | HT2379 | 366 | IVL, involucrin | CTTAAGCAGGAGAAA[A/G]CACAAAGGGATCAGC | M | A | G | T | A |
| G4106a3 | WI-18356 | HT2379 | 416 | IVL, involucrin | AGAAGAGAAGAAGCT[C/T]TTAGACCAGCAACTG | S | C | T | L | L |
| G4112a3 | WI-18357 | HT4401 | 853 | KIF5A, kinesin family | GGTGGACCTGGCAGG[G/A]AGTGAGAAGGTCAGC | S | G | A | G | G |
| | | | | member 5A | | | | | | |
| G4112a4 | WI-18358 | HT4401 | 859 | KIF5A, kinesin family | CCTGGCAGGGAGTGA[G/A]AAGGTCAGCAAGACT | S | G | A | E | E |
| | | | | member 5A | | | | | | |
| G4112a5 | WI-18359 | HT4401 | 3103 | KIF5A, kinesin family | CACATCTTCTGGCGG[C/T]CCCTTGGCTTCCTAC | S | C | T | G | G |
| | | | | member 5A | | | | | | |
| G4114a2 | WI-18360 | HT4160 | 812 | fibrinogen-like protein | AGGCACGTCTCGATG[G/A]GAGCACCAACTTCAC | M | G | A | G | E |
| | | | | pT49, ? | | | | | | |
| G4122a2 | WI-18362 | HT97538 | 1209 | myosin-I, ? | CGGAGCACCACGGTT[C/T]TCGGGCTCCTGGATA | M | C | T | L | F |
| G4122a3 | WI-18363 | HT97538 | 2437 | myosin-I, ? | GGCGGCAGCTGCCCC[G/A]GAATGTCCTGGACAC | M | G | A | R | Q |
| G4122a4 | WI-18364 | HT97538 | 2609 | myosin-I, ? | TGAGATCTTCAAGGG[C/A]AAGAAGGATAATTAC | S | C | A | G | G |
| G4122a5 | WI-18365 | HT97538 | 2745 | myosin-I, ? | CCTGTTGTGAAATAC[G/A]ACCGCAAGGGCTACA | M | G | A | D | N |
| G4122a6 | WI-18366 | HT97538 | 3119 | myosin-I, ? | CAAGAACGGGCACCT[G/T]GCTGTGGTCGCCCCA | S | G | T | L | L |
| G4124a3 | WI-18367 | HT0925 | 787 | TGM3, transglutaminase 3 (E | GCCGGGACCCAAGGA[G/A]CTGGGACGGCAGCGT | M | G | A | S | N |
| | | | | polypeptide, protein- | | | | | | |
| | | | | glutamine-gamma- | | | | | | |
| | | | | glutamyltransferase) | | | | | | |
| G4161a1 | WI-18371 | HT0853 | 977 | KNS2, kinesin 2 (60-70kD) | TGCCCCTCTGCAAGC[A/C]GGCCCTGGAGGACCT | M | A | C | Q | P |
| G4218a2 | WI-18380 | HT1681 | 614 | phosphatidyl-inositol | ACCGTGTCTCTTTGT[G/A]ATACAAACCACATCA | M | G | A | D | N |
| | | | | glycan, class A, ? | | | | | | |
| G4227a4 | WI-18381 | HT1929 | 1027 | proteoglycan 2, ? | GGGTGCCAGACCTGC[C/T]GCTACCTCCTGGTGA | M | C | T | R | C |
| G4255a5 | WI-18382 | HT2907 | 566 | CRYAB, crystallin, alpha B | GAGTTCCACAGGAAA[T/C]ACCGGATCCCAGCTG | M | T | C | Y | H |
| G439a1 | WI-18431 | M67454 | 377 | TNFRSF6, tumor necrosis | CCAATTCTGCCATAA[G/A]CCCTGTCCTCCAGGT | S | G | A | K | K |
| | | | | factor receptor superfamily, | | | | | | |
| | | | | member 6 | | | | | | |
| G439a2 | WI-18432 | M67454 | 416 | TNFRSF6, tumor necrosis | AGCTAGGGACTGCAC[A/G]GTCAATGGGGATGAA | S | A | G | T | T |
| | | | | factor receptor superfamily, | | | | | | |
| | | | | member 6 | | | | | | |
| G4406a4 | WI-18298 | HT3564 | 901 | ACPP, acid phosphatase, | CGCATGACACTACTG[T/C]GACTGGCCTACAGAT | M | T | C | V | A |
| | | | | prostate | | | | | | |
| G441a5 | WI-18478 | M77349 | 267 | TGFBI, transforming growth | TACCAAAGGAAAATC[T/C]GTGGCAAATCAACAG | M | T | C | C | R |
| | | | | factor, beta-induced, 68kD | | | | | | |
| G411a6 | WI-18479 | M77349 | 984 | TGFBI, transforming growth | AACAACCACATCTTG[A/G]AGTCAGCTATGTGTG | M | A | G | K | E |
| | | | | factor, beta-induced, 68kD | | | | | | |
| G441a7 | WI-18480 | M77349 | 927 | TGFBI, transforming growth | CCTAGTGAGACTTTG[A/G]ACCGTATCCTGGGCG | M | A | G | N | D |
| | | | | factor, beta-induced, 68kD | | | | | | |
| G441a8 | WI-18519 | M77349 | 581 | TGFBI, transforming growth | CCATATGGTGGGCAG[G/A]CGAGTCCTGACTGAT | S | G | A | R | R |
| | | | | factor, beta-induced, 68kD | | | | | | |
| G441a9 | WI-18520 | M77349 | 708 | TGFBI, transforming growth | CGGCTCCTGAAAGCC[G/A]ACCACCATGCAACCA | M | G | A | D | N |
| | | | | factor, beta-induced, 68kD | | | | | | |
| G441a10 | WI-18521 | M77349 | 820 | TGFBI, transforming growth | TTGAGACCCTTCGGG[C/T]TGCTGTGGCTGCATC | M | C | T | A | V |
| | | | | factor, beta-induced, 68kD | | | | | | |
| G411a11 | WI-18524 | M77349 | 1640 | TGFBI, transforming growth | ACTGACGGAGACCCT[C/T]AACCGGGAAGGAGTC | S | C | T | L | L |
| | | | | factor, beta-induced, 68kD | | | | | | |
| G441a12 | WI-18963 | M77349 | 2148 | TGFBI, transforming growth | GCTCTCCGCCAATTT[C/T]TCTCAGATTTCCACA | - | C | T | | |
| | | | | factor, beta-induced, 68kD | | | | | | |
| G4411a3 | WI-18299 | HT97468 | 1387 | acyl-CoA, ? | ACACAGTGTTGTCCC[G/A]AGCGCCGGGAGGCGT | - | G | A | | |
| G4417a17 | WI-18690 | HT0542 | 1238 | AOAH, acyloxyacyl hydrolase | AAATCTATTTACCTT[C/A]GCTTATGGAAAAGAA | M | C | A | R | S |
| | | | | (neutrophil) | | | | | | |
| G4417a18 | WI-18691 | HT0542 | 2088 | AOAH, acyloxyacyl hydrolase | CTATGGGGGCTGCCA[C/T]GTCACAGGCCCAAAG | - | C | T | | |
| | | | | (neutrophil) | | | | | | |
| G442a5 | WI-18455 | M94582 | 1262 | IL8RA, interleukin 8 | TGTGGTCACAGGAAG[C/T]AGAGGAGGCCACGTT | - | C | T | | |
| | | | | receptor, alpha | | | | | | |
| G442a6 | WI-18644 | M94582 | 696 | IL8RA, interleukin 8 | TGTTACGGATCCTGC[C/T]CCAGTCCTTTGGCTT | M | C | T | P | L |
| | | | | receptor, alpha | | | | | | |
| G442a7 | WI-18645 | M94582 | 789 | IL8RA, interleukin 8 | CCCACATGGGGCAGA[A/G]GCACCGGGCCATGCG | M | A | G | K | R |
| | | | | receptor, alpha | | | | | | |
| G442a8 | WI-18646 | M94582 | 825 | IL8RA, interleukin 8 | TCTTTGCTGTCGTCC[T/C]CATCTTCCTGCTTTG | M | T | C | L | P |
| | | | | receptor, alpha | | | | | | |
| G442a9 | WI-18647 | M94582 | 838 | IL8RA, interleukin 8 | CCTCATCTTCCTGCT[T/C]TGCTGGCTGCCCTAC | S | T | C | L | L |
| | | | | receptor, alpha | | | | | | |
| G442a10 | WI-19273 | M94582 | 140 | IL8RA, interleukin 8 | TACAGCTCTACCCTG[C/T]CCCCTTTTCTACTAG | M | C | T | P | S |
| | | | | receptor, alpha | | | | | | |
| G442a11 | WI-19274 | M94582 | 210 | IL8RA, interleukin 8 | AGTATTTTGTGGTCA[T/C]TATCTATGCCCTGGT | M | T | C | I | T |
| | | | | receptor, alpha | | | | | | |
| G442a12 | WI-19275 | M94582 | 430 | IL8RA, interleukin 8 | GGTCTCACTCCTGAA[G/A]GAAGTCAACTTCTAT | S | G | A | K | K |
| | | | | receptor, alpha | | | | | | |
| G4428a3 | WI-18179 | HT97524 | 1017 | ADFP, adipose | TGTACCACAGAACAT[C/T]CAAGATCAAGCCAAG | S | C | T | I | I |
| | | | | differentiation-related | | | | | | |
| | | | | protein; adipophilin | | | | | | |
| G4442a4 | WI-18695 | HT2326 | 2651 | ALD, | CCGGCCCCTGCCCCG[C/T]CCCCAAGCTCGGATC | - | C | T | | |
| | | | | adrenoleukodystrophy/adrenom | | | | | | |
| | | | | yeloneuropathy | | | | | | |
| G445a2 | WI-18477 | U40373 | 916 | Human cell surface | GGCTTTGATTCTTGC[A/G]GTTTGCATTGCAGTC | S | A | G | A | A |
| | | | | glycoprotein CD44 mRNA, | | | | | | |
| | | | | complete cds., ? | | | | | | |
| G4451a1 | WI-18696 | HT0365 | 215 | AKR1A1, aldo-keto reductase | CTGCTATCTACGGCA]A/G]TGAGCCTGAGATTGG | M | A | G | N | S |
| | | | | family 1, member A1 | | | | | | |
| | | | | (aldehyde reductase) | | | | | | |
| G4455a1 | WI-18697 | HT0580 | 1124 | ALDOB, aldolase B, fructose- | TATGAAGCGGGCCAT[G/A]GCTAACTGCCAGGCG | M | G | A | M | I |
| | | | | bisphosphate | | | | | | |
| G4456a2 | WI-18698 | HT0626 | 496 | ALDOC, aldolase C, fructose- | TCAAGGGCTGGATGG[G/A]CTCTCAGAACGCTGT | S | G | A | G | G |
| | | | | bisphosphate | | | | | | |
| G446a9 | WI-18648 | U64198 | 1354 | IL12RB2, interleukin 12 | ATTTCAAAAGGCTTC[C/T]GTGAGCAGATGTACC | S | C | T | S | S |
| | | | | receptor, beta 2 | | | | | | |
| G446a10 | WI-18649 | U64198 | 2245 | IL12RB2, interleukin 12 | CACAGAGGAAAAGGG[G/A]AGCATTTTAATTTCA | S | G | A | G | G |
| | | | | receptor, beta 2 | | | | | | |
| G446a11 | WI-18650 | U64198 | 2962 | IL12RB2, interleukin 12 | GCTGGAGAGCAGGGG[C/T]TCCGACCCAAAGCCA | S | C | T | G | G |
| | | | | receptor, beta 2 | | | | | | |
| G446a12 | WI-18651 | U64198 | 2977 | IL12RB2, interleukin 12 | CTCCGACCCAAAGCC[A/C]GAAAACCCAGCCTGT | S | A | C | P | P |
| | | | | receptor, beta 2 | | | | | | |
| G446a13 | WI-18652 | U64198 | 2997 | IL12RB2, interleukin 12 | ACCCAGCCTGTCCCT[G/A]GACGGTGCTCCCAGC | N | G | A | W | * |
| | | | | receptor, beta 2 | | | | | | |
| G446a14 | WI-19276 | U64198 | 909 | IL12RB2, interleukin 12 | TCAATTCTCAAGTCA[C/T]AGGTCTTCCCCTTGG | M | C | T | T | I |
| | | | | receptor, beta 2 | | | | | | |
| G446a15 | WI-19277 | U64198 | 1711 | IL12RB2, interleukin 12 | GGCAAGAGGAAAAAT[T/C]CTCCACTATCAGGTG | S | T | C | I | I |
| | | | | receptor, beta 2 | | | | | | |
| G4468a2 | WI-18325 | HT4305 | 1149 | FUT7, fucosyltransferase 7 | GGTTGGTTTCAGGCC[T/A]GAGATCCGCTGGCCG | N | T | A | * | R |
| | | | | (alpha (1,3) | | | | | | |
| | | | | fucosyltransferase) | | | | | | |
| G4468a3 | WI-18326 | HT4305 | 1157 | FUT7, fucosyltransferase 7 | TCAGGCCTGAGATCC[G/C]CTGGCCGGGGGAGGT | - | G | C | | |
| | | | | (alpha (1,3) | | | | | | |
| | | | | fucosyltransferase) | | | | | | |
| G4473a4 | WI-18327 | HT1352 | 400 | FUCA1, fucosidase, alpha-L- | GGCGCCAAGTATGTA[G/T]TTTTGACGACAAAGC | M | G | T | V | F |
| | | | | 1, tissue | | | | | | |
| G4488a5 | WI-18181 | HT1559 | 684 | SLC4A2, solute carrier | AGGAGGCGGAGGCGG[A/T]GGCGGTGGCGGTGGC | M | A | T | E | V |
| | | | | family 4, anion exchanger, | | | | | | |
| | | | | member 2 (erythrocyte | | | | | | |
| | | | | membrane protein band 3-like | | | | | | |
| | | | | 1) | | | | | | |
| G4488a6 | WI-18182 | HT1559 | 702 | SLC4A2, solute carrier | CGGTGGCGGTGGCCA[G/C]TGGCACAGCAGGGGG | M | G | C | S | T |
| | | | | family 4, anion exchanger, | | | | | | |
| | | | | member 2 (erythrocyte | | | | | | |
| | | | | membrane protein band 3-like | | | | | | |
| | | | | 1) | | | | | | |
| G4488a7 | WI-18329 | HT1559 | 3837 | SLC4A2, solute carrier | GAGGGACCGATGGAC[G/A]AGGGGACAGGCTGGT | - | G | A | | |
| | | | | family 4, anion exchanger, | | | | | | |
| | | | | member 2 (erythrocyte | | | | | | |
| | | | | membrane protein band 3-like | | | | | | |
| | | | | 1) | | | | | | |
| G450A2 | WI-18503 | X85740 | 649 | CCR4, chemokine (C-C motif) | TGACTTATGGGGTCA[T/C]CACCAGTTTGGCTAC | M | T | C | I | T |
| | | | | receptor 4 | | | | | | |
| G450a3 | WI-18677 | X85740 | 1111 | CCR4, chemokine (C-C motif) | TTTTTCTGGGGGAGA[A/T]ATTTCGCAAGTACAT | M | A | T | K | I |
| | | | | receptor 4 | | | | | | |
| G4502a14 | WI-18334 | HT4840 | 269 | ASS, argininosuccinate | TCATTGAGGATGTCA[G/T]CAGGGAGTTTGTGGA | M | G | T | S | I |
| | | | | synthetase | | | | | | |
| G4502a15 | WI-18335 | HT4840 | 1227 | ASS, argininosuccinate | GCAGGGTGATTATGA[G/T]CCAACTGATGCCACC | M | G | T | E | D |
| | | | | synthetase | | | | | | |
| G4526a3 | WI-18538 | HT4994 | 672 | ATP5D, ATP synthase, H+ | AGCTCCTGGGGTCCC[G/C]GCCACCTGGGGAAGC | - | G | C | | |
| | | | | transporting, mitochondrial | | | | | | |
| | | | | F1 complex, delta subunit | | | | | | |
| G4548a4 | WI-18539 | HT1574 | 3814 | ATPase, Ca2+ transporting, | TTAGCTGAGGACCCT[C/G]TCGCCTGCCCGCCCG | - | C | G | | |
| | | | | plasma membrane, isoform 2, | | | | | | |
| | | | | ? | | | | | | |
| G4548a5 | WI-18701 | HT1574 | 3427 | ATPase, Ca2+ transporting, | GGAGATCGACCACGC[G/A]GAGCGGGAGCTGCGG | S | G | A | A | A |
| | | | | plasma membrane, isoform 2, | | | | | | |
| | | | | ? | | | | | | |
| G4549a5 | WI-18186 | HT1346 | 1519 | ATP2B4, ATPase, Ca++ | CATGTCTGCTCTCAC[G/A]GTTTTCATCCTGATT | S | G | A | T | T |
| | | | | transporting, plasma | | | | | | |
| | | | | membrane 4 | | | | | | |
| G4549a6 | WI-18187 | HT1346 | 1612 | ATP2B4, ATPase, Ca++ | CATCTACATCCAGTA[C/T]TTTGTCAAGTTCTTC | S | C | T | Y | Y |
| | | | | transporting, plasma | | | | | | |
| | | | | membrane 4 | | | | | | |
| G4549a7 | WI-18188 | HT1346 | 2317 | ATP2B4, ATPase, Ca++ | CCGGACTATCTGCAT[A/G]GCTTACCGGGACTTC | M | A | G | I | M |
| | | | | transporting, plasma | | | | | | |
| | | | | membrane 4 | | | | | | |
| G4549a8 | WI-18189 | HT1346 | 2596 | ATP2B4, ATPase, Ca++ | CCGGCTCATCCGCAA[C/T]GAGAAAGGCGAGGTA | S | C | T | N | N |
| | | | | transporting, plasma | | | | | | |
| | | | | membrane 4 | | | | | | |
| G4549a9 | WI-18190 | HT1346 | 4067 | ATP2B4, ATPase, Ca++ | TTTCCATTTTCGTCT[G/A]TCCCATCTATGAGGT | - | G | A | | |
| | | | | transporting, plasma | | | | | | |
| | | | | membrane 4 | | | | | | |
| G4549a10 | WI-18191 | HT1346 | 4101 | ATP2B4, ATPase, Ca++ | GATGGGACTTTTCAT[C/T]GTCACGTCAGCTGCT | - | C | T | | |
| | | | | transporting, plasma | | | | | | |
| | | | | membrane 4 | | | | | | |
| G4549a11 | WI-18540 | HT1346 | 2983 | ATP2B4, ATPase, Ca++ | AGCCTTCACTGGAGC[C/T]TGTATCACTCAGGAT | S | C | T | A | A |
| | | | | transporting, plasma | | | | | | |
| | | | | membrane 4 | | | | | | |
| G4549a12 | WI-18541 | HT1346 | 3805 | ATP2B4, ATPase, Ca++ | GACCCACCCTGAATT[C/T]GCCATAGAGGAGGAG | S | C | T | F | F |
| | | | | transporting, plasma | | | | | | |
| | | | | membrane 4 | | | | | | |
| G4593a6 | WI-18303 | HT97373 | 1207 | BARD1, BRCA1 associated | TGGTACATCAGGGAG[G/C]AAAAACAGTAACATG | M | G | C | R | S |
| | | | | RING domain 1 | | | | | | |
| G4593a7 | WI-18304 | HT97373 | 1252 | BARD1, BRCA1 associated | TAGTCTTTCACCAGG[T/G]ACACCACCTTCTACA | S | T | G | G | G |
| | | | | RING domain 1 | | | | | | |
| G4593a8 | WI-18692 | HT97373 | 2045 | BARD1, BRCA1 associated | ATTCCTGAAGGTCCA[C/T]GCAGAAGCAGGCTCA | M | C | T | R | C |
| | | | | RING domain 1 | | | | | | |
| G4597a2 | WI-18693 | HT4270 | 254 | CDH11, cadherin 11 (OB- | GGGGCACCTGCGGCC[C/T]TCCTTCCATGGGCAC | S | C | T | P | P |
| | | | | cadherin, osteoblast) | | | | | | |
| G4597a3 | WI-18694 | HT4270 | 919 | CDH11, cadherin 11 (OB- | GGACAACCAAAGTGA[C/T]GATCACACTGACCGA | M | C | T | T | M |
| | | | | cadherin, osteoblast) | | | | | | |
| G4598a1 | WI-18966 | HT4271 | 295 | CDH12, cadherin 12 (N- | GTGCTGGAAGAATAC[G/A]TGGGCTCCGAGCCTC | M | G | A | V | M |
| | | | | cadherin 2) | | | | | | |
| G4599a2 | WI-18967 | HT4273 | 2520 | CDH13, cadherin 13, H- | GGACTGCAACGCGGC[G/A]GGGGCCCTGCGCTTC | S | G | A | A | A |
| | | | | cadherin (heart) | | | | | | |
| G4601a1 | WI-18968 | HT4274 | 617 | CDH4, cadherin 4, R- | CAAAGACAATGACAT[C/T]CCCATCCGGTACAGC | S | C | T | I | I |
| | | | | cadherin (retinal) | | | | | | |
| G4601a2 | WI-18969 | HT4274 | 824 | CDH4, cadherin 4, R- | CTACGTCATCGACAT[G/A]AATGACAACCACCCT | M | G | A | M | I |
| | | | | cadherin (retinal) | | | | | | |
| G4601a3 | WI-18970 | HT4274 | 875 | CDH4, cadherin 4, R- | CAACTGCTCCGTGGA[C/T]GAGGGCTCCAAGCCA | S | C | T | D | D |
| | | | | cadherin (retinal) | | | | | | |
| G4603a1 | WI-18971 | HT4275 | 176 | CDH8, cadherin 8 | CCAAAAGAGGCTGGG[T/C]TTGGAATCAAATGTT | M | T | C | V | A |
| G4603a2 | WI-18972 | HT4275 | 481 | CDH8, cadherin 8 | AATGACAATGCACCA[G/C]AGTTTGTTAATGGAC | M | G | C | E | Q |
| G4606a1 | WI-18815 | HT27350 | 1923 | CDH6, cadherin 6, K- | CATGCAATCCTGCCA[T/C]GCGGAGGCGCTCATC | S | T | C | H | H |
| | | | | cadherin (fetal kidney) | | | | | | |
| G4606a2 | WI-18816 | HT27350 | 2136 | CDH6, cadherin 6, K- | GGACACCCAGGCTTT[T/C]GATATCGGCACCCTG | S | T | C | F | F |
| | | | | cadherin (fetal kidney) | | | | | | |
| G4606a3 | WI-18973 | HT27350 | 2396 | CDH6, cadherin 6, K- | AGTCAGTGACCACGG[A/C]TGCAGATCAAGACTA | M | A | C | D | A |
| | | | | cadherin (fetal kidney) | | | | | | |
| G4614a6 | WI-18699 | HT4835 | 209 | S100A3, S100 calcium- | GCTGCAGAAGGAGCT[G/A]GCCACCTGGACCCCG | S | G | A | L | L |
| | | | | binding protein A3 | | | | | | |
| G4614a7 | WI-18700 | HT4835 | 453 | S100A3, S100 calcium- | CACACCCCCTCCTAC[C/T]CTCTCTCCTGTACCC | - | C | T | | |
| | | | | binding protein A3 | | | | | | |
| G4644a10 | WI-18542 | HT1736 | 1148 | CPS1, carbamoyl-phosphate | TATGCCTTGGACAAC[A/G]CCCTCCCTGCTGGCT | M | A | G | T | A |
| | | | | synthetase 1, mitochondrial | | | | | | |
| G4644a11 | WI-18543 | HT1736 | 1150 | CPS1, carbamoyl-phosphate | TGCCTTGGACAACAC[C/T]CTCCCTGCTGGCTGG | S | C | T | T | T |
| | | | | synthetase 1, mitochondrial | | | | | | |
| G4674a5 | WI-18330 | HT1393 | 1838 | CDC25B, cell division cycle | CAGCTGCCCTATGGG[C/T]CTGCCGGGCTGAGGG | - | C | T | | |
| | | | | 25B | | | | | | |
| G4691a13 | WI-18336 | HT97602 | 234 | CMKBR9, chemokine (C-C | GGTCTTGCTCCGTTA[C/T]GTGCCTCGCAGGCGG | S | C | T | Y | Y |
| | | | | motif) receptor 9 | | | | | | |
| G4691a14 | WI-18337 | HT97602 | 680 | CMKBR9, chemokine (C-C | GGTTTCTCCTTCCAC[T/C]CCTTGCCATGATCTT | M | T | C | L | P |
| | | | | motif) receptor 9 | | | | | | |
| G4726a6 | WI-18975 | HT48614 | 1146 | AOC3, amine oxidase, copper | GTGTCCAGGGAAGTC[G/A]AGTGGCCTCCTCACT | M | G | A | R | Q |
| | | | | containing 3 (vascular | | | | | | |
| | | | | adhesion protein 1) | | | | | | |
| G4726a7 | WI-18976 | HT48614 | 1437 | AOC3, amine oxidase, copper | CCCCCAAGACAATAC[G/A]TGATGCCTTTTGTGT | M | G | A | R | H |
| | | | | containing 3 (vascular | | | | | | |
| | | | | adhersion protein 1) | | | | | | |
| G4726a8 | WI-18977 | HT48614 | 1481 | AOC3, amine oxidase, copper | CAGGGCCTCCCCCTG[C/T]GGCGACACCACTCAG | M | C | T | R | W |
| | | | | containing 3 (vascular | | | | | | |
| | | | | adhesion protein 1) | | | | | | |
| G4732a1 | WI-18978 | HT48529 | 1697 | DOCK1, dedicator of cyto- | CTATAAGGCCGAAGC[G/A]AAGAAGCTGGAAGAT | S | G | A | A | A |
| | | | | kinesis 1 | | | | | | |
| G4732a2 | WI-18979 | HT48529 | 2667 | DOCK1, dedicator of cyto- | CTGGAGGCCTGCTGT[C/T]AGCTGCTCAGCCACA | N | C | T | Q | * |
| | | | | kinesis 1 | | | | | | |
| G4732a3 | WI-18980 | HT48529 | 2792 | DOCK1, dedicator of cyto- | CATTTCCATGGGACG[A/G]GATTCTGAACTCATT | S | A | G | R | R |
| | | | | kinesis 1 | | | | | | |
| G4732a4 | WI-18981 | HT48529 | 3374 | DOCK1, dedicator of cyto- | GTGTGAATTCCATTC[G/A]ACCCGAAGCTTCCAA | S | G | A | S | S |
| | | | | kinesis 1 | | | | | | |
| G4732a5 | WI-18982 | HT48529 | 3398 | DOCK1, dedicator of cyto- | CTTCCAAATGTTTGA[A/T]AATGAGATCATCACC | M | A | T | E | D |
| | | | | kinesis 1 | | | | | | |
| G4732a6 | WI-18983 | HT48529 | 4211 | DOCK1, dedicator of cyto- | CGACGATATTAAAAA[C/T]TCTCCTGGCCAGTAT | S | C | T | N | N |
| | | | | kinesis 1 | | | | | | |
| G4732a7 | WI-18984 | HT48529 | 4505 | DOCK1, dedicator of cyto- | CCTGGAGAATGCCAT[C/T]GAGACCATGCAGCTG | S | C | T | I | I |
| | | | | kinesis 1 | | | | | | |
| G4732a8 | WI-18985 | HT48529 | 5345 | DOCK1, dedicator of cyto- | TCCAGTTACACCAAG[A/G]GCCAAGCTCAGCTTC | S | A | G | R | R |
| | | | | kinesis 1 | | | | | | |
| G4732a9 | WI-18986 | HT48529 | 5400 | DOCK1, dedicator of cyto- | AACGGCATGACGGGG[G/A]CGGACGTGGCCGATG | M | G | A | A | T |
| | | | | kinesis 1 | | | | | | |
| G4732a10 | WI-18987 | HT48529 | 5558 | DOCK1, dedicator of cyto- | GCCCAGCAAAACTCC[G/A]CCTCCTCCCCCTCCA | S | G | A | P | P |
| | | | | kinesis 1 | | | | | | |
| G4732a11 | WI-18988 | HT48529 | 5592 | DOCK1, dedicator of cyto- | ACAACTCGCAAGCAG[A/G]CATCGGTGGACTCTG | M | A | G | T | A |
| | | | | kinesis 1 | | | | | | |
| G4732a12 | WI-18989 | HT48529 | 5606 | DOCK1, dedicator of cyto- | GACATCGGTGGACTC[T/C]GGGATCGTGCAGTGA | S | T | C | S | S |
| | | | | kinesis 1 | | | | | | |
| G4732a13 | WI-18990 | HT48529 | 5623 | DOCK1, dedicator of cyto- | GGATCGTGCAGTGAC[A/G]TCGCAAGGCTCTCTG | - | A | G | | |
| | | | | kinesis 1 | | | | | | |
| G4732a14 | WI-18991 | HT48529 | 5631 | DOCK1, dedicator of cyto- | CAGTGACATCGCAAG[G/C]CTCTCTGGAAAGAGT | - | G | C | | |
| | | | | kinesis 1 | | | | | | |
| G4754a1 | WI-18397 | HT1855 | 1047 | CYP2C8, cytochrome P450 | ACATGCCTTACACTG[A/G]TGCTGTAGTGCACGA | M | A | G | D | G |
| | | | | subfamily IIC (mephenytoin 4- | | | | | | |
| | | | | hydroxylase), polypeptide 8 | | | | | | |
| G4788a4 | WI-18354 | HT28249 | 1875 | DSC3, desmocollin 3 | ATCCTGATGAACCTG[T/C]CCATGGAGCTCCATT | M | T | C | V | A |
| G4827a2 | WI-18183 | HT97477 | 223 | elongation, ? | AGCTCCAGCGGGTCC[C/G]CGGCAAACTCCTTCC | M | C | G | P | A |
| G4827a3 | WI-18184 | HT97477 | 489 | elongation, ? | CCAGCAGTGGAAGGG[C/A]GCCTCCAACTACGTG | S | C | A | G | G |
| G4828a1 | WI-18702 | HT4894 | 170 | elongation factor Ts, | CACAAGGAGGCCCAG[A/T]AGGAGGGCTGGAGCA | N | A | T | K | * |
| | | | | mitochondrial, ? | | | | | | |
| G4828a2 | WI-18703 | HT4894 | 201 | elongation factor Ts, | AAGCTGCCAAGCTCC[A/G]AGGGAGGAAGACCAA | M | A | G | Q | R |
| | | | | mitochondrial, ? | | | | | | |
| G4828a3 | WI-18704 | HT4894 | 334 | elongation factor Ts, | GGTCCAGCAAGTAGC[C/T]CTTGGAACCATGATG | S | C | T | A | A |
| | | | | mitochondrial, ? | | | | | | |
| G5110a1 | WI-18919 | HT3433 | 1916 | HK2, hexokinase 2 | CATGGATAAGCTACA[A/T]ATCAAAGACAAGAAG | M | A | T | Q | H |
| G5110a2 | WI-18920 | HT3433 | 2243 | HK2, hexokinase 2 | CATGGTGGAAGGCGA[T/C]GAGGGGCGGATGTGT | S | T | C | D | D |
| G5110a3 | WI-18921 | HT3433 | 2452 | HK2, hexokinase 2 | AGGAGCTGCTCTTTG[G/C]GGGGAAGCTCAGCCC | M | G | C | G | A |
| G5110a4 | WI-18922 | HT3433 | 2594 | HK2, hexokinase 2 | GACTCAGGAGGACTG[C/T]GTGGCCACTCACCGG | S | C | T | C | C |
| G5110a5 | WI-18923 | HT3433 | 2649 | HK2, hexokinase 2 | TCCGCCAGCCTGTGC[G/T]CAGCCACCCTGGCCG | M | G | T | A | S |
| G5110a6 | WI-18924 | HT3433 | 2980 | HK2, hexokinase 2 | AGGTAGAAATGGAGC[G/A]AGGTCTGAGCAAGGA | M | G | A | R | Q |
| G5110a7 | WI-18925 | HT3433 | 3566 | HK2, hexokinase 2 | GGAGGAGATGCGCAA[C/T]GTGGAACTGGTGGAA | S | C | T | N | N |
| G5110a8 | WI-18926 | HT3433 | 3698 | HK2, hexokinase 2 | GCTTTCACTCAACCC[C/G]GGCAAGCAGAGGTTC | S | C | G | P | P |
| G5110a9 | WI-18927 | HT3433 | 3788 | HK2, hexokinase 2 | CACCAAGCGTGGACT[A/G]CTCTTCCGAGGCCGC | S | A | G | L | L |
| G5110a10 | WI-18928 | HT3433 | 4021 | HK2, hexokinase 2 | CCGCTGTGGTGGACA[G/A]GATACGAGAAAACCG | M | G | A | R | K |
| G5188a1 | WI-18740 | HT33638 | 1144 | interferon-related protein | GGGCATGCACCACCA[C/T]CTCCAGAACAATGAG | S | C | T | H | H |
| | | | | SM15, ? | | | | | | |
| G5188a2 | WI-18741 | HT36638 | 1311 | interferon-related protein | GTGTGCGGGACAAGC[G/A]GGCAGACATCCTGTG | M | G | A | R | Q |
| | | | | SM15, ? | | | | | | |
| G5191a1 | WI-18904 | HT3774 | 2395 | interleukin-2 receptor, | GAAGGGGTCGCACCT[C/T]TCTCACAGGCCCCCT | M | C | T | L | F |
| | | | | alpha chain, kappa B binding | | | | | | |
| | | | | protein, ? | | | | | | |
| G5191a2 | WI-18905 | HT3774 | 3015 | interleukin-2 receptor, | GGGGCCAGTGAAGGG[C/A]GTGTTTGACAAGGAG | S | C | A | G | G |
| | | | | alpha chain, kappa B binding | | | | | | |
| | | | | protein, ? | | | | | | |
| G5191a3 | WI-18906 | HT3774 | 3729 | interleukin-2 receptor, | CAGTTCTCAGGCTGC[C/T]GCCCGGGTCGTGAGC | S | C | T | A | A |
| | | | | alpha chain, kappa B binding | | | | | | |
| | | | | protein, ? | | | | | | |
| G5191a4 | WI-18907 | HT3774 | 4629 | interleukin-2 receptor, | TGCCACGATCCGCAT[C/T]GTGCAGGGACTGGGA | S | C | T | I | I |
| | | | | alpha chain, kappa B binding | | | | | | |
| | | | | protein, ? | | | | | | |
| G5213a1 | WI-18213 | HT4528 | 168 | CDKN1B, cyclin-dependent | CATGGAAGAGGCGAG[C/T]CAGCGCAAGTGGAAT | S | C | T | S | S |
| | | | | kinase inhibitor 1B (p27, | | | | | | |
| | | | | Kip1) | | | | | | |
| G5213a2 | WI-18214 | HT4528 | 326 | CDKN1B, cyclin-dependent | AGGAGAGCCAGGATG[T/G]CAGCGGGAGCCGCCC | M | T | G | V | G |
| | | | | kinase inhibitor 1B (p27, | | | | | | |
| | | | | Kip1) | | | | | | |
| G5217a1 | WI-18932 | HT3714 | 5845 | LCT, lactase | AGTTTCTTCATCTAT[C/G]TTTACCGGCCACCAA | — | C | G | | |
| G5235a1 | WI-18898 | HT2457 | 160 | SPN, sialophorin (gpL115, | CTCTGGGGAGCACAA[C/T]AGCAGTGCAGACACC | M | C | T | T | I |
| | | | | leukosialin, CD43) | | | | | | |
| G5235a2 | WI-18899 | HT2457 | 372 | SPN, sialophorin (gpL115, | CCTTTACCTGAGCCA[A/G]CAACCTACCAGGAAG | M | A | G | T | A |
| | | | | leukosialin, CD43) | | | | | | |
| G5235a3 | WI-18900 | HT2457 | 932 | SPN, sialophorin (gpL115, | CCTGCTGTGGCGCCG[G/A]CGGCAGAAGCGGCGG | S | G | A | R | R |
| | | | | leukosialin, CD43) | | | | | | |
| G5235a4 | WI-18901 | HT2457 | 974 | SPN, sialophorin (gpL115, | CGTGCTGAGCAGAGG[C/T]GGCAAGCGTAACGGG | S | C | T | G | G |
| | | | | leukosialin, CD43) | | | | | | |
| G5235a5 | WI-18902 | HT2457 | 1110 | SPN, sialophorin (gpL115, | GAGGGGTCTAGCCGT[C/G]GGCCCACGCTCACCA | M | C | G | R | G |
| | | | | leukosialin, CD43) | | | | | | |
| G5235a6 | WI-18903 | HT2457 | 1231 | SPN, sialophorin (gpL115, | AGCCACTGGTGGCCA[G/C]TGAGGATGGGGCTGT | M | G | C | S | T |
| | | | | leukosialin, CD43) | | | | | | |
| G5237a1 | WI-18933 | HT3964 | 641 | SORD, sorbitol | TGCCTGCAGGAGAGG[C/T]GGAGTTACCCTGGGA | S | C | T | G | G |
| | | | | dehydrogenase | | | | | | |
| G5237a2 | WI-18934 | HT3964 | 672 | SORD, sorbitol | CACAAGGTCCTTGTG[T/C]GTGGAGCTGGGCCAA | M | T | C | C | R |
| | | | | dehydrogenase | | | | | | |
| G5237a3 | WI-18935 | HT3964 | 827 | SORD, sorbitol | CAAGGAGAGCCCTCA[G/A]GAAATCGCCAGGAAA | S | G | A | Q | Q |
| | | | | dehydrogenase | | | | | | |
| G5237a4 | WI-18936 | HT3964 | 853 | SORD, sorbitol | GGAAAGTAGAAGGTC[T/A]GCTGGGGTGCAAGCC | M | T | A | L | Q |
| | | | | dehydrogenase | | | | | | |
| G5237a5 | WI-18937 | HT3964 | 914 | SORD, sorbitol | GGCCTCCATCCAGGC[G/A]GGCATCTACGCCACT | S | G | A | A | A |
| | | | | dehydrogenase | | | | | | |
| G5237a6 | WI-18938 | HT3964 | 943 | SORD, sorbitol | CTCGCTCTGGTGGGA[C/A]CCTCGTGCTTGTGGG | M | C | A | T | N |
| | | | | dehydrogenase | | | | | | |
| G5254a1 | WI-18577 | HT1581 | 235 | BSG, basigin | TGGCTGAAGGGGGGC[G/T]TGGTGCTGAAGGAGG | M | G | T | V | L |
| G5254a2 | WI-18578 | HT1581 | 252 | BSG, basigin | GGTGCTGAAGGAGGA[C/T]GCGCTGCCCGGCCAG | S | C | T | D | D |
| G5254a3 | WI-18579 | HT1581 | 291 | BSG, basigin | GTTCAAGGTGGACTC[C/G]GACGACCAGTGGGGA | S | C | G | S | S |
| G5254a4 | WI-18580 | HT1581 | 384 | BSG, basigin | TCCCAGAGTGAAGGC[C/T]GTGAAGTCGTCAGAA | S | C | T | A | A |
| G5254a5 | WI-18581 | HT1581 | 429 | BSG, basigin | GGAGACGGCCATGCT[G/A]GTCTGCAAGTCAGAG | S | G | A | L | L |
| G5254a6 | WI-18582 | HT1581 | 898 | BSG, basigin | GACGCTCCCTGCTCC[G/A]CGTCTGCGCCGCCGC | — | G | A | | |
| G5256a1 | WI-18892 | HT2001 | 170 | CAPG, capping protein | CAAGAGAACCAGGGC[G/A]TCTTCTTCTCGGGGG | M | G | A | V | I |
| | | | | (actin filament), gelsolin- | | | | | | |
| | | | | like | | | | | | |
| G5256a2 | WI-18893 | HT2001 | 307 | CAPG, capping protein | TGTGCACCTCAACAC[G/A]CTGCTGGGAGAGCGG | S | G | A | T | T |
| | | | | (actin filament), gelsolin- | | | | | | |
| | | | | like | | | | | | |
| G5256a3 | WI-18894 | HT2001 | 862 | CAPG, capping protein | CGCTGACTCCAGCCC[C/A]TTTGCCCTTGAACTG | S | C | A | P | P |
| | | | | (actin filament), gelsolin- | | | | | | |
| | | | | like | | | | | | |
| G5257a1 | WI-18895 | HT27995 | 204 | macrophage differentiation- | ATTCCTCATTGTTCC[G/A]GCCATCGTGGGCAGT | S | G | A | P | P |
| | | | | associated protein, ? | | | | | | |
| G5257a2 | WI-18896 | HT27995 | 219 | macrophage differentiation- | GGCCATCGTGGGCAG[T/C]GCCCTCCTCCATCGG | S | T | C | S | S |
| | | | | associated protein, ? | | | | | | |
| G5257a3 | WI-18897 | HT27995 | 609 | macrophage differentiation- | AATGAACAACACCGA[T/C]GGACTTCAGGAACTT | S | T | C | D | D |
| | | | | associated protein, ? | | | | | | |
| G5333a1 | WI-18593 | HT97206 | 307 | FUT8, fucosyltransferase 8 | TGAACGCTTAAAACA[G/A]CAGAATGAAGACTTG | S | G | A | Q | Q |
| | | | | (alpha (1,6) | | | | | | |
| | | | | fucosyltransfease) | | | | | | |
| G5333a2 | WI-18594 | HT97206 | 443 | FUT8, fucosyltransferase 8 | CAGATTGAAAATTAC[A/C]AGAAACAGACCAGAA | M | A | C | K | Q |
| | | | | (alpha (1,6) | | | | | | |
| | | | | fucosyltransferase) | | | | | | |
| G538a11 | WI-19456 | M55531 | 677 | SLC2A5, solute carrier | TGGGGCTGACCGGGG[T/A]CCCCGCGGCGCTGCA | M | T | A | V | D |
| | | | | family 2 (facilitated | | | | | | |
| | | | | glucose transporter), member | | | | | | |
| | | | | 5 | | | | | | |
| G5418a1 | WI-18948 | HT3037 | 260 | PBP, prostatic binding | GGTTAAGAATAGACC[C/T]ACCAGCATTTCGTGG | S | C | T | P | P |
| | | | | protein | | | | | | |
| G5418a2 | WI-18949 | HT3037 | 394 | PBP, prostatic binding | TCAACATGAAGGGCA[A/G]TGACATCAGCAGTGG | M | A | G | N | S |
| | | | | protein | | | | | | |
| G5418a3 | WI-18950 | HT3037 | 164 | PBP, prostatic binding | CCTGCAAGAAGTGGA[C/T]GAGCAGCCGCAGCAC | S | C | T | D | D |
| | | | | protein | | | | | | |
| G5437a1 | WI-18599 | HT27771 | 126 | PGD, phosphogluconate | TGTCTCCAAAGTTGA[C/T]GATTTCTTGGCCAAT | S | C | T | D | D |
| | | | | dehydrogenase | | | | | | |
| G5437a2 | WI-18600 | HT27771 | 738 | PGD, phosphogluconate | TCTCAAGTTCCAAGA[C/T]ACCGATGGCAAACAC | S | C | T | D | D |
| | | | | dehydrogenase | | | | | | |
| G5437a3 | WI-18601 | HT27771 | 742 | PGD, phosphogluconate | AAGTTCCAAGACACC[G/A]ATGGCAAACACCTGC | M | G | A | D | N |
| | | | | dehydrogenase | | | | | | |
| G5475a1 | WI-18908 | HT97315 | 542 | ?, ? | AAAGCTGTGCTTGAT[G/A]GACTTGATGTGCTCC | M | G | A | G | R |
| G5475a2 | WI-18909 | HT97315 | 559 | ?, ? | ACTTGATGTGCTCCT[T/C]GCCCAGGAGGTTCGC | S | T | C | L | L |
| G5475a3 | WI-18910 | HT97315 | 1001 | ?, ? | AAATTTTCTCCTTAC[C/T]TGGGCCAGATGATTA | S | C | T | L | L |
| G5475a4 | WI-18911 | HT97315 | 1022 | ?, ? | CAGATGATTAATCTG[C/T]GTAGACTCCTCCTCT | M | C | T | R | C |
| G5475a5 | WI-18912 | HT97315 | 1498 | ?, ? | TTCCATCTCCATATC[T/C]GCCTTGCAGAGTCTC | S | T | C | S | S |
| G5475a6 | WI-18913 | HT97315 | 1762 | ?, ? | CTGTTTCATGCCTAA[C/T]TAGCTGGGTGCACAT | S | C | T | N | N |
| G5479a1 | WI-18951 | HT0761 | 594 | prosaposin, ? | CCTCAGGACGGCCCC[C/T]GCAGCAAGCCCCAGC | M | C | T | R | C |
| G5479a2 | WI-18952 | HT0761 | 608 | prosaposin, ? | CCGCAGCAAGCCCCA[G/T]CCAAAGGATAATGGG | M | G | T | Q | H |
| G5479a3 | WI-18953 | HT0761 | 1490 | prosaposin, ? | TGGAGCCTGCCCCTC[G/A]GCCCATAAGCCCTTG | S | G | A | S | S |
| G5487a1 | WI-18939 | HT97615 | 478 | PI12, protease inhibitor 12 | ATGAAAAAATATTTT[A/T]ATGCAGCAGTAAATC | M | A | T | N | Y |
| | | | | (neuroserpin) | | | | | | |
| G5487a2 | WI-18940 | HT97615 | 657 | PI12, protease inhibitor 12 | GGGGAACTGGAAGTC[G/A]CAGTTTAGGCCTGAA | S | G | A | S | S |
| | | | | (neuroserpin) | | | | | | |
| G5497a1 | WI-18587 | HT1286 | 1624 | ?, ? | AACAACAAGGGACCC[G/A]TCAAGGTCGTGGTGG | M | G | A | V | I |
| G5498a1 | WI-18215 | HT4254 | 830 | GSK3B, glycogen synthase | GGGATAGTGGTGTGG[A/G]TCAGTTGGTAGAAAT | M | A | G | D | G |
| | | | | kinase 3 beta | | | | | | |
| G5554a1 | WI-18941 | HT4883 | 1225 | PTPRJ, protein tyrosine | GAAGGTGGCTTGGAT[G/A]CCAGCAATACAGAGA | M | G | A | M | I |
| | | | | phosphatase, receptor type, | | | | | | |
| | | | | J | | | | | | |
| G5554a2 | WI-18942 | HT4883 | 1326 | PTPRJ, protein tyrosine | CCGGCCCAGCAGTCCC[G/A]AGACACGGAAGTCCT | M | G | A | R | Q |
| | | | | phosphatase, receptor type, | | | | | | |
| | | | | J | | | | | | |
| G5554a3 | WI-18943 | HT4883 | 1463 | PTPRJ, protein tyrosine | ATTCAGGTTTTTGAC[G/A]TCACCGCTGTGAACA | M | G | A | V | I |
| | | | | phosphatase, receptor type, | | | | | | |
| | | | | J | | | | | | |
| G5554a4 | WI-18944 | HT4883 | 2219 | PTPRJ, protein tyrosine | TCCACTGCACAGTAC[A/G]CACGGCCCAGCAATG | M | A | G | T | A |
| | | | | phosphatase, receptor type, | | | | | | |
| | | | | J | | | | | | |
| G5554a5 | WI-18945 | HT4883 | 2289 | PTPRJ, protein tyrosine | CTTTAAGTTGGCAGA[A/T]CTTTGATGACGCCTC | M | A | T | N | I |
| | | | | phosphatase, receptor type, | | | | | | |
| | | | | J | | | | | | |
| G5554a6 | WI-18946 | HT4883 | 3997 | PTPRJ, protein tyrosine | CACTGACCTGCTCAT[C/T]AACTTCCGGTACCTC | S | C | T | I | I |
| | | | | phosphatase, receptor type, | | | | | | |
| | | | | J | | | | | | |
| G5554a7 | WI-18947 | HT4883 | 4321 | PTPRJ, protein tyrosine | CTATGAAAACCTTGC[G/A]CCCGTGACCACATTT | S | G | A | A | A |
| | | | | phosphatase, receptor type, | | | | | | |
| | | | | J | | | | | | |
| G5613a1 | WI-18589 | HT97193 | 664 | rhodanese, ? | GACTCGGGCCATATC[C/T]GTGGTGCCGTCAACA | M | C | T | R | C |
| G5613a2 | WI-18590 | HT97193 | 816 | rhodanese, ? | AGTCACCGCCTGCCA[C/T]GTGGCCTTGGCTGCC | S | C | T | H | H |
| G5638a1 | WI-18608 | HT3181 | 709 | SHMT2, serine | CAAGGTGATTCCCTC[G/A]CCTTTCAAGCACGCG | S | G | A | S | S |
| | | | | hydroxymethyltransferase 2 | | | | | | |
| | | | | (mitochondrial) | | | | | | |
| G5638a2 | WI-18609 | HT3181 | 724 | SHMT2, serine | GCCTTTCAAGCACGC[G/A]GACATCTGCACCACC | S | G | A | A | A |
| | | | | hydroxymethyltransferase 2 | | | | | | |
| | | | | (mitochondrial) | | | | | | |
| G5638a3 | WI-18610 | HT3181 | 880 | SHMT2, serine | GCTGTTCCCATCCCT[T/G]CAGGGGGGCCCCCAC | S | T | G | L | L |
| | | | | hydroxymethyltransferase 2 | | | | | | |
| | | | | (mitochondrial) | | | | | | |
| G5638a4 | WI-18761 | HT3181 | 1267 | SHMT2, serine | TATAGATGAAGGGGT[C/T]AACATTGGCTTAGAG | S | C | T | V | V |
| | | | | hydroxymethyltransferase 2 | | | | | | |
| | | | | (mitochondrial) | | | | | | |
| G5639a1 | WI-18611 | HT4498 | 1965 | SRPK1, SFRS protein kinase | GAAGTATGAGTGGTC[T/G]CAGGAAGAGGCAGCT | S | T | G | S | S |
| | | | | 1 | | | | | | |
| G5663a1 | WI-18606 | HT4409 | 1831 | RAB8IP, Rab8 interacting | GACACCAAAGGCTGC[T/C]TGCAGTGTCGTGTGG | S | T | C | L | L |
| | | | | protein (GC kinase) | | | | | | |
| G5663a2 | WI-18606 | HT4409 | 2606 | RAB8IP, Rab8 interacting | CCAGGCCCTGGCCCT[G/T]CTGGGGCTGAAGGTC | — | G | T | | |
| | | | | protein (GC kinase) | | | | | | |
| G5664a1 | WI-18753 | HT2748 | 1130 | CDK6, cyclin-dependent | TTAAGCTGATCCTGC[G/A]GAGAACACCCTTGGT | — | G | A | | |
| | | | | kinase 6 | | | | | | |
| G5678a1 | WI-18763 | HT4978 | 498 | sialyltransferase, SThM, ? | TATCCGGTGTGCCGT[G/C]GTGGGCAACGGAGGC | S | G | C | V | V |
| G5678a2 | WI-18764 | HT4978 | 527 | sialyltransferase, SThM, ? | GCATTCTGAATGGGT[C/T]CCGCCAGGGTCCCAA | M | C | T | S | F |
| G570a1 | WI-19120 | L13288 | 538 | ?, ? | GACCGGCTACACCAT[C/T]GGCTACGGCCTGTCC | S | C | T | I | I |
| G570a2 | WI-19121 | L13288 | 894 | ?, ? | GCTGGGGGGTACCCA[G/A]CACATTCACCATGGT | M | G | A | S | N |
| G570a4 | WI-19399 | L13288 | 1278 | ?, ? | TCCTCAATGGTGAGG[T/C]GCAGGCGGAGCTGAG | M | T | C | V | A |
| G570a5 | WI-19400 | L13288 | 1308 | ?, ? | GGCGGAAGTGGCGGC[G/A]CTGGCACCTGCAGGG | M | G | A | R | H |
| G570a6 | WI-19401 | L13288 | 1354 | ?, ? | CCCCAAATACCGGCA[C/T]CCGTCGGGAGGCAGC | S | C | T | H | H |
| G5709a5 | WI-18246 | HT3731 | 820 | SMPD1, sphingomyelin | CGGAGCCCTGTGGCA[C/T]GCCCTGCCGTCTGGC | M | C | T | T | M |
| | | | | phosphodiesterase 1, acid | | | | | | |
| | | | | lysosomal (acid | | | | | | |
| | | | | sphingomyelinase) | | | | | | |
| G5788a1 | WI-18556 | HT1698 | 163 | EIF4A1, eukaryotic | CCGTGGCATCTACGC[C/G]TATGGTTTTGAGAAG | S | C | G | A | A |
| | | | | translation initiation | | | | | | |
| | | | | factor 4A, isoform 1 | | | | | | |
| G5788a2 | WI-18557 | HT1698 | 283 | EIF4A1, eukaryotic | CACATTTGCCATATC[G/A]ATTCTGCAGCAGATT | S | G | A | S | S |
| | | | | translation initiation | | | | | | |
| | | | | factor 4A, isoform 1 | | | | | | |
| G5790a1 | WI-18874 | HT3679 | 1663 | EIF4B, eukaryotic | TAGCCGTGGTCCAGG[A/C]GACGGAGGGAACAGA | S | A | C | G | G |
| | | | | translation initiation | | | | | | |
| | | | | factor 4B | | | | | | |
| G5817a1 | WI-18591 | HT0288 | 1825 | tumor necrosis factor alpha- | TCCAGCACTTCTGCA[C/T]CCAGCAACGGCTCCCC | M | C | T | T | I |
| | | | | inducible primary response | | | | | | |
| | | | | gene B94, ? | | | | | | |
| G5817a2 | WI-18592 | HT0288 | 1835 | tumor necrosis factor alpha- | CTGCACCCAGCACGG[C/T]TCCCCGGCGACCTGG | S | C | T | G | G |
| | | | | inducible primary response | | | | | | |
| | | | | gene B94, ? | | | | | | |
| G5817a3 | WI-18747 | HT0288 | 2151 | tumor necrosis factor alpha- | GCCTTGGGCACACCC[C/T]GCTGGGAGCTGTTAA | — | C | T | | |
| | | | | inducible primary response | | | | | | |
| | | | | gene B94, ? | | | | | | |
| G5836a2 | WI-18777 | HT1549 | 892 | CSK, c-src tyrosine kinase | GGTGCAGCTCCTGGG[C/T]GTGATCGTGGAGGAG | S | C | T | G | G |
| G5836a3 | WI-18778 | HT1549 | 925 | CSK, c-src tyrosine kinase | GGGCGGGCTCTACAT[C/T]GTCACTGAGTACATG | S | C | T | I | I |
| G5836a4 | WI-18779 | HT1549 | 974 | CSK, c-src tyrosine kinase | GACTACCTGCGGTCT[A/C]GGGGTCGGTCAGTGC | S | A | C | R | R |
| G5869a1 | WI-18954 | HT0929 | 3985 | ITK, IL2-inducible T-cell | ACCAGCCCAGGACCC[T/C]CCAGAGGCAGCCTGG | — | T | C | | |
| | | | | kinase | | | | | | |
| G5869a2 | WI-18955 | HT0929 | 4036 | ITK, IL2-inducible T-cell | CACCATGGAAGCAGC[A/C]TCCTGACCACAGCTG | — | A | C | | |
| | | | | kinase | | | | | | |
| G5870a1 | WI-18956 | HT3217 | 1038 | PTPN2, protein tyrosine | AGAAGAAAAACTGAC[A/C]GGTGACCGATGTACA | S | A | C | T | T |
| | | | | phosphatase, non-receptor | | | | | | |
| | | | | type 2 | | | | | | |
| G5908a1 | WI-18549 | HT1444 | 798 | UCHL1, ubiquitin carboxyl- | TTCTGCAGACACGCC[T/C]TCCCCTCAGCCACAC | — | T | C | | |
| | | | | terminal esterase L1 | | | | | | |
| | | | | (ubiquitin thiolesterase) | | | | | | |
| G5909a1 | WI-18878 | HT0284 | 494 | ubiqiutin carrier protein E2- | CTACGAGGAGTATGC[G/A]GCTCGGGCCCGTCTG | S | G | A | A | A |
| | | | | EPF, ? | | | | | | |
| G5909a2 | WI-18879 | HT0284 | 507 | ubiquitin carrier protein E2- | GCGGCTCGGGCCCGT[C/T]TGCTCACAGAGATCC | S | C | T | L | L |
| | | | | EPF, ? | | | | | | |
| G5909a3 | WI-18880 | HT0284 | 586 | ubiquitin carrier protein E2- | TGGCCAGTGGCACTG[A/C]AGCTTCCTCCACCGA | M | A | C | E | A |
| | | | | EPF, ? | | | | | | |
| G5909a4 | WI-18881 | HT0284 | 615 | ubiquitin carrier protein E2- | GACCCTGGGGCCCCA[G/T]GGGGCCCGGGAGGGG | M | G | T | G | W |
| | | | | EPF, ? | | | | | | |
| G5909a5 | WI-18882 | HT0284 | 622 | ubiquitin carrier protein E2- | GGGCCCCAGGGGGCC[C/T]GGGAGGGGCTGAGGG | M | C | T | P | L |
| | | | | EPF, ? | | | | | | |
| G5909a6 | WI-18883 | HT0284 | 623 | ubiquitin carrier protein E2- | GGCCCCAGGGGGCCC[G/A]GGAGGGGCTGAGGGT | S | G | A | P | P |
| | | | | EPF, ? | | | | | | |
| G5909a7 | WI-18884 | HT0284 | 563 | ubiquitin carrier protein E2- | CAGGGCCGAAGCCGG[T/G]CGGGCCCTGGCCAGT | S | T | G | G | G |
| | | | | EPF, ? | | | | | | |
| G5922a1 | WI-18929 | HT0037 | 513 | Unknown protein product | AGGTGGTGACCTGCA[G/A]AAAGCAGGAAAGCTC | S | G | A | Q | Q |
| | | | | CIT987SK-A-2A8_1, ? | | | | | | |
| G5922a2 | WI-18930 | HT0037 | 1798 | Unknown protein product | GCTATGGATGTTCAA[C/T]TTGTGTGGGAAATAC | M | C | T | L | F |
| | | | | CIT987SK-A-2A8_1, ? | | | | | | |
| G5922a3 | WI-18931 | HT0037 | 2568 | Unknown protein product | GAAAGCTGTTTTGGC[T/C]GAAAGTTATGAAAAA | S | T | C | A | A |
| | | | | CIT987SK-A-2A8_1, ? | | | | | | |
| G607a1 | WI-19562 | HT33636 | 665 | MAPKAPK3, mitogen-activated | GATTTTGGCTTTGCT[A/G]AGGAGACCACCCAAA | M | A | G | K | E |
| | | | | protein kinase-activated | | | | | | |
| | | | | protein kinase 3 | | | | | | |
| G6091a1 | WI-18583 | HT97327 | 377 | cell, ? | AGAAGCATGTTTATT[G/A]CTTCAGAATAAGCAC | M | G | A | C | Y |
| G6091a2 | WI-18584 | HT97327 | 580 | cell, ? | GCTGATGAGGATGAC[C/T]GGGAAATTTATGATA | M | C | T | R | W |
| G6110a1 | WI-18567 | HT1126 | 835 | CD81, CD81 antigen (target | CGATGACCTCTTCTC[C/T]GGGAAGCTGTACCTC | S | C | T | S | S |
| | | | | of antiproliferative | | | | | | |
| | | | | antibody 1) | | | | | | |
| G6110a2 | WI-18568 | HT1126 | 877 | CD81, CD81 antigen (target | TGCCATCGTGGTCGC[T/C]GTGATCATGATCTTC | S | T | C | A | A |
| | | | | of antiproliferative | | | | | | |
| | | | | antibody 1) | | | | | | |
| G6112a1 | WI-18570 | HT5011 | 397 | RANGAP1, Ran GTPase | GGTGTGCAAGGCTTC[G/C]AGGCCCTGCTCAAGA | M | G | C | E | Q |
| | | | | activating protein 1 | | | | | | |
| G6112a2 | WI-18571 | HT5011 | 696 | RANGAP1, Ran GTPase | CCTGGCCCAGGCTTT[C/T]GCTGTCAACCCCCTG | S | C | T | F | F |
| | | | | activating protein 1 | | | | | | |
| G6112a3 | WI-18572 | HT5011 | 870 | RANGAP1, Ran GTPase | AGATGCCATCCGCGG[C/T]GGCCTGCCCAAGCTA | S | C | T | G | G |
| | | | | activating protein 1 | | | | | | |
| G5112a4 | WI-18573 | HT5011 | 1201 | RANGAP1, Ran GTPase | GAAGAGCCTCAGCAG[C/G]GAGGGCAGGGAGAGA | M | C | G | R | G |
| | | | | activating protein 1 | | | | | | |
| G6112a5 | WI-18574 | HT5011 | 1548 | RANGAP1, Ran GTPase | CTTCCTCACCAGGCT[C/G]CTCGTGCACATGGGT | S | C | G | L | L |
| | | | | activating protein 1 | | | | | | |
| G619a1 | WI-19151 | HT2549 | 1281 | calcineurin A1, ? | AAAGTGACAGAAATG[T/C]TGGTAAATGTTCTGA | S | T | C | L | L |
| G6373a1 | WI-18564 | HT28143 | 219 | H3FA, H3 histone family, | CCAGCGCCTAGTGCG[C/T]GAGATTGCGCAGGAC | S | C | T | R | R |
| | | | | member A | | | | | | |
| G6381a1 | WI-18885 | HT28122 | 207 | H4FG, H4 histone family, | GAACGTTATTCGAGA[C/T]GCCGTCACCTATACG | S | C | T | D | D |
| | | | | member G | | | | | | |
| G6381a2 | WI-18886 | HT28122 | 105 | H4FG, H4 histone family, | TACAAAACCGGCTAT[C/T]CGCCGTTTGGCTCGG | S | C | T | I | I |
| | | | | member G | | | | | | |
| G651a1 | WI-18236 | HT5206 | 234 | ?, ? | CCTCATTGCCTCCTT[T/C]TCACACCGATCCATT | S | T | C | F | F |
| G6766a1 | WI-18887 | HT2641 | 918 | major centromere autoantigen | CTCGGGCCTGCGGCA[T/C]GTGCAGCTGGCCTTC | S | T | C | H | H |
| | | | | CENP-B, ? | | | | | | |
| G6766a2 | WI-18888 | HT2641 | 1208 | major centromere autoantigen | GTGAGGGAGAGGAAG[A/G]GGAGGAGGAGGAGGA | M | A | G | E | G |
| | | | | CENP-B, ? | | | | | | |
| G683a4 | WI-18399 | Y08723 | 419 | BMP1, bone morphogenetic | GGGTCATCCCCTTTG[T/C]CATTGGGGGAAACTT | M | T | C | V | A |
| | | | | protein 1 | | | | | | |
| G683a5 | WI-18400 | Y08723 | 544 | BMP1, bone morphogenetic | TATATTGTGTTCACC[T/C]ATCGACCTTGAGGGT | M | T | C | Y | H |
| | | | | protein 1 | | | | | | |
| G6839a1 | WI-18889 | HT97463 | 789 | non-histone, ? | TGTCTGCTAAACCAG[C/T]TCCTCCAAAACCAGA | M | C | T | A | V |
| G6839a2 | WI-18890 | HT97463 | 889 | non-histone, ? | TGCTGGAAAGGATGG[A/G]AACAACCCTGCAAAA | S | A | G | G | G |
| G6839a3 | WI-18891 | HT97463 | 953 | non-histone, ? | GCGGAAGGCACTGGG[G/A]ATGCCAAGTGAAATG | M | G | A | D | N |
| G7000a1 | WI-18738 | HT0116 | 1316 | MYCL2, v-myc avian | AGTAGATTGCAGAAT[C/G]GATTGCAGCCAGTGC | — | C | G | | |
| | | | | myelocytomatosis viral | | | | | | |
| | | | | oncogene homolog 2 | | | | | | |
| G7000a2 | WI-18739 | HT0116 | 1317 | MYCL2, v-myc avian | GTAGATTGCAGAATC[G/C]ATTGCAGCCAGTGCA | — | G | C | | |
| | | | | myelocytomatosis viral | | | | | | |
| | | | | oncogene homolog 2 | | | | | | |
| G7086a1 | WI-18558 | HT27382 | 1748 | DDX8, DEAD/H (Asp-Glu-Ala- | ACCCAGATGTCAATC[C/T]TTGAGCAGAGGGAGA | M | C | T | L | F |
| | | | | Asp/His) box polypeptide 8 | | | | | | |
| | | | | (RNA helicase) | | | | | | |
| G7086a2 | WI-18559 | HT27382 | 3340 | DDX8, DEAD/H (Asp-Glu-Ala- | CATAATGGACAGACA[C/T]AAGCTGGATGTTGTT | S | C | T | H | H |
| | | | | Asp/His) box polypeptide 8 | | | | | | |
| | | | | (RNA helicase) | | | | | | |
| G7087a1 | WI-18560 | HT1506 | 502 | DDX5, DEAD/H (Asp-Glu-Ala- | TGTCATGGATGTTAT[T/A]GCAAGACAGAATTTC | S | T | A | I | I |
| | | | | Asp/His) box polypeptide 5 | | | | | | |
| | | | | (RNA helicase, 68kD) | | | | | | |
| G7087a2 | WI-18561 | HT1506 | 1613 | DDX5, DEAD/H (Asp-Glu-Ala- | GTCGAAGACAGAGGT[T/G]CAGGTCGTTCCAGGG | M | T | G | S | A |
| | | | | Asp/His) box polypeptide 5 | | | | | | |
| | | | | (RNA helicase, 68kD) | | | | | | |
| G7088a1 | WI-18562 | HT33614 | 424 | RNA polymerase II, ? | TGAGTAGGGGCCAGA[G/A]GGGGCTCTGCTCGGC | — | G | A | | |
| G7088a2 | WI-18563 | HT33614 | 436 | RNA polymerase II, ? | AGAGGGGGCTCTGCT[C/T]GGCCTGTGAGCCCCG | — | C | T | | |
| G7183a1 | WI-18553 | HT27991 | 2107 | ?, ? | CCCAATGCCCCCTGT[G/T]CATCCCCCACCTCCC | S | G | T | V | V |
| G719a1 | WI-18394 | X16468 | 4006 | COL2A1, collagen, type II, | CTGGACGAAGCAGCT[G/A]GCAACCTCAAGAAGG | M | G | A | G | S |
| | | | | alpha 1 (primary | | | | | | |
| | | | | osteoarthritis, | | | | | | |
| | | | | spondyloepiphyseal | | | | | | |
| | | | | dysplasia, congenital) | | | | | | |
| G7192a1 | WI-18875 | HT4462 | 1454 | SFRS8, splicing factor, | CAAGTGCACTTGCCC[C/T]CGTGGCCGCCATCAT | M | C | T | P | L |
| | | | | arginine/serine-rich 8 | | | | | | |
| | | | | (suppressor-of-white- | | | | | | |
| | | | | apricot, Drosophila homolog) | | | | | | |
| G7192a2 | WI-18876 | HT4462 | 1473 | SFRS8, splicing factor, | GGCCGCCATCATCCC[C/T]CCGCCCCCCGACGTC | S | C | T | P | P |
| | | | | arginine/serine-rich 8 | | | | | | |
| | | | | (suppressor-of-white- | | | | | | |
| | | | | apricot, Drosophila homolog) | | | | | | |
| G7192a3 | WI-18877 | HT4462 | 2831 | SFRS8, splicing factor, | GCTCCAGCCAGGAGC[G/A]CTCCAGGGGAGTCTC | M | G | A | R | H |
| | | | | arginine/serine-rich 8 | | | | | | |
| | | | | (suppressor-of -white- | | | | | | |
| | | | | apricot, Drosophila homolog) | | | | | | |
| G722a8 | WI-18274 | HT3162 | 1500 | COL4A2, collagen, type IV, | GGGCTCCTGCCTGGC[G/A]CGGTTCAGCACCATG | S | G | A | A | A |
| | | | | alpha 2 | | | | | | |
| G722a9 | WI-18275 | HT3162 | 1756 | COL4A2, collagen, type IV, | TGGATCGGATATTCC[T/C]TCCTCATGCACACGG | M | T | C | F | L |
| | | | | alpha 2 | | | | | | |
| G722a10 | WI-18276 | HT3162 | 1173 | COL4A2, collagen, type IV, | CGGAGAACCAGGTTT[T/C]CGTGGGGCTCCAGGG | S | T | C | F | F |
| | | | | alpha 2 | | | | | | |
| G722a11 | WI-18277 | HT3162 | 1283 | COL4A2, collagen, type IV, | GGCCGATTGGCCAAG[A/C]AGGTGCACCAGGCCG | M | A | C | E | A |
| | | | | alpha 2 | | | | | | |
| G722a12 | WI-18278 | HT3162 | 1398 | COL4A2, collagen, type IV, | GGAGCCCATGTGCCC[G/A]GTGGGCATGAACAAA | S | G | A | P | P |
| | | | | alpha 2 | | | | | | |
| G7224a1 | WI-18914 | HT2862 | 261 | PLS3, plastin 3 (T isoform) | GAGAGAAATTATTCA[G/T]AAACTCATGCTGGAT | M | G | T | Q | H |
| G7224a2 | WI-18915 | HT2862 | 1329 | PLS3, plastin 3 (T isoform) | TCTTGGTGTCAATCC[T/C]CACGTAAACCATCTC | S | T | C | P | P |
| G7224a3 | WI-18916 | HT2862 | 1381 | PLS3, plastin 3 (T isoform) | CTGGTAATCTTACAG[T/C]TATATGAACGAATTA | S | T | C | L | L |
| G7224a4 | WI-18917 | HT2862 | 1522 | PLS3, plastin 3 (T isoform) | GCTAAATTCTCCCTG[G/A]TTGGCATTGGAGGGC | M | G | A | V | I |
| G7224a5 | WI-18918 | HT2862 | 1537 | PLS3, plastin 3 (T isoform) | GTTGGCATTGGAGGG[C/G]AAGACCTGAATGATG | M | C | G | Q | E |
| G759a1 | WI-18398 | U08032 | 250 | SULT1A1, sulfotransferase | GTACGGGTGCCCTTC[C/T]TTGAGGTCAATGATC | M | C | T | L | F |
| | | | | family 1A, phenol- | | | | | | |
| | | | | preferring, member 1 | | | | | | |
| G804a16 | WI-18818 | Z26653 | 130 | LAMA2, laminin, alpha 2 | GCAGCGGCCGCAGCA[G/C]CAGCGGCAGTCACAG | M | G | C | Q | H |
| | | | | (merosin, congenital | | | | | | |
| | | | | muscular dystrophy) | | | | | | |
| G804a17 | WI-18819 | Z26653 | 205 | LAMA2, laminin, alpha 2 | TTCTAATGCTCTTAT[C/T]ACGACCAATGCAACA | S | C | T | I | I |
| | | | | (merosin, congenital | | | | | | |
| | | | | muscular dystrophy) | | | | | | |
| G804a18 | WI-18820 | Z26653 | 2143 | LAMA2, laminin, alpha 2 | GATGGATGCCATCTT[C/T]AGGTTGAGCTCTGTT | S | C | T | F | F |
| | | | | (merosin, congenital | | | | | | |
| | | | | muscular dystrophy) | | | | | | |
| G804a19 | WI-18823 | Z26653 | 3662 | LAMA2, laminin, alpha 2 | GAGGCTCTGCAGCAC[A/G]CGACCACCAAGGGCA | M | A | G | T | A |
| | | | | (merosin, congenital | | | | | | |
| | | | | muscular dystrophy) | | | | | | |
| G804a20 | WI-18829 | Z26653 | 7809 | LAMA2, laminin, alpha 2 | GACAGGCCTATTATG[T/C]AATACTCCTCAACAG | M | T | C | V | A |
| | | | | (merosin, congenital | | | | | | |
| | | | | muscular dystrophy) | | | | | | |
| G804a21 | WI-18830 | Z26653 | 7879 | LAMA2, laminin, alpha 2 | AATGAGGAAAATTGT[C/G]ATCAGACCAGAGCCG | S | C | G | V | V |
| | | | | (merosin, congenital | | | | | | |
| | | | | muscular dystrophy) | | | | | | |
| G804a22 | WI-18831 | Z26653 | 7894 | LAMA2, laminin, alpha 2 | CATCAGACCAGAGCC[G/AAATCTGTTTCATGAT | S | G | A | P | P |
| | | | | (merosin, congenital | | | | | | |
| | | | | muscular dystrophy) | | | | | | |
| G804a23 | WI-18832 | Z26653 | 7955 | LAMA2, laminin, alpha 2 | ACTAGAGGCATCTTT[A/G]CAGTTCAAGTGGATG | M | A | G | T | A |
| | | | | (merosin, congenital | | | | | | |
| | | | | muscular dystrophy) | | | | | | |
| G804a24 | WI-18873 | Z26653 | 5883 | LAMA2, laminin, alpha 2 | AAGTTGCCAAAGAAG[C/T]CAAAGATCTTGCACA | M | C | T | A | V |
| | | | | (merosin, congenital | | | | | | |
| | | | | muscular dystrophy) | | | | | | |
| G8089a1 | WI-18387 | U39550 | 1004 | Homo sapiens UDP- | ATATGATCTCTACAG[T/C]CACACATCAATTTGG | S | T | C | S | S |
| | | | | glucuronosyltransferase | | | | | | |
| | | | | (UGT1J) gene, exon 1, | | | | | | |
| | | | | partial cds., ? | | | | | | |
| G8089a2 | WI-18388 | U39550 | 977 | Homo sapiens UDP- | TGAAATTCTCCAAAC[C/A]CCTGTCACGGCATAT | S | C | A | T | T |
| | | | | glucuronosyltransferase | | | | | | |
| | | | | (UGT1J) gene, exon 1, | | | | | | |
| | | | | partial cds., ? | | | | | | |
| G8089a3 | WI-18389 | U39550 | 983 | Homo sapiens UDP- | TCTCCAAACCCCTGT[C/T]ACGGCATATGATCTC | S | C | T | V | V |
| | | | | glucuronosyltransferase | | | | | | |
| | | | | (UGT1J) gene, exon 1, | | | | | | |
| | | | | partial cds., ? | | | | | | |
| G8157a1 | WI-18227 | AF084644 | 1160 | NR1I2, nuclear receptor | GCTGAAATTCCACTA[C/T]ATGCTGAAGAAGCTG | S | C | T | Y | Y |
| | | | | subfamily 1, group I, member | | | | | | |
| | | | | 2 | | | | | | |
| G83a8 | WI-18180 | HT1576 | 4317 | DNMT1, DNA (cytosine-5-)- | CTGGCGCGATCTGCC[C/T]AACATCGAGGTGCGG | S | C | T | P | P |
| | | | | methyltransferase 1 | | | | | | |
| G840a4 | WI-18411 | L13858 | 2159 | SOS1, son of sevenless | TCATTTCAAGTGTAA[G/A]AGGGAAAGCTATGAA | M | G | A | R | K |
| | | | | (Drosophila) homolog 1 | | | | | | |
| G8675a1 | WI-18245 | NM_002039 | 1251 | ?, ? | GTTACTGTATCCCTA[C/T]AGCAGGGATGTCGCC | M | C | T | T | I |
| G8675a2 | WI-18620 | NM_002039 | 2011 | ?, ? | GGAATACTTAGATCT[C/T]GACTTAGATTCTGGG | S | C | T | L | L |
| G8697a1 | WI-18244 | U65065 | 3006 | ?, ? | CTCGAGGGGAGCCCC[C/T]ACCCCACGGATGTTG | — | C | T | | |
| G898a5 | WI-18279 | X96783 | 1051 | SYT5, synaptotagmin 5 | CTTCGCCTTCAAGGT[C/A]CCCTACGTGGAGCTG | S | C | A | V | V |
| G898a6 | WI-18280 | X96783 | 1078 | SYT5, synaptotagmin 5 | GCTGGGGGGCAGGGT[G/A]CTGGTCATGGCGGTG | S | G | A | V | V |
| G898a7 | WI-18281 | X96783 | 1142 | SYT5, synaptotagmin 5 | ATCGGGGAGGTGCGG[G/A]TCCCTATGAGCTCCG | M | G | A | V | I |
| G898a8 | WI-18282 | X96783 | 1271 | SYT5, synaptotagmin 5 | GTCCCCACGGCCGGG[A/G]AGCTCACCGTCATCG | M | A | G | K | E |
| G909a1 | WI-18237 | HT3173 | 189 | DNM1, dynamin 1 | GGTGGGCGGCCAGAG[C/T]GCCGGCAAGAGCTCG | S | C | T | S | S |
| G909a2 | WI-18238 | HT3173 | 378 | DNM1, dynamin 1 | TGAGATCGAGGCCGA[G/A]ACCGACAGGGTCACC | S | G | A | E | E |
| G909a3 | WI-18239 | HT3173 | 423 | DNM1, dynamin 1 | CATCTCGCCGGTGCC[T/C]ATCAACCTCCGCGTC | S | T | C | P | P |
| G957a23 | WI-19441 | HT3419 | 190 | calcium channel, voltage, | CGGCAGAACTGTTTC[A/G]CCGTCAACAGATCCC | — | A | G | | |
| | | | | gated, alpha 1E subunit, | | | | | | |
| | | | | alt. transcript 2, ? | | | | | | |
| G957a24 | WI-19442 | HT3419 | 2574 | calcium channel, voltage- | GTCCCTCAAGGGGGA[T/A]GGAGGGGACCGATCC | M | T | A | D | E |
| | | | | gated, alpha 1E subunit, | | | | | | |
| | | | | alt. transcript 2, ? | | | | | | |
| G957a25 | WI-19443 | HT3419 | 3444 | calcium channel, voltage- | GGCCTGCCACTACAT[C/T]GTGAACCTGCGCTAC | S | C | T | I | I |
| | | | | gated, alpha 1E subunit, | | | | | | |
| | | | | alt. transcript 2, ? | | | | | | |
| G957a26 | WI-19444 | HT3419 | 3455 | calcium channel, voltage- | ACATCGTGAACCTGC[G/C]CTACTTTGAGATGTG | M | G | C | R | P |
| | | | | gated, alpha 1E subunit, | | | | | | |
| | | | | alt. transcript 2, ? | | | | | | |
| G957a27 | WI-19543 | HT3419 | 1308 | calcium channel, voltage- | CTGTGTTGATATCTC[C/G]TCTGTGGGCACACCT | — | C | G | | |
| | | | | gated, alpha 1E subunit, | | | | | | |
| | | | | alt. transcript 2, ? | | | | | | |
| G957a28 | WI-19544 | HT3419 | 2809 | calcium channel, voltage- | TCCTCTTCAGCCTCC[C/T]GGAGCAGGTCTGCCA | M | C | T | R | W |
| | | | | gated, alpha 1E subunit, | | | | | | |
| | | | | alt. transcript 2, ? | | | | | | |
| G957a29 | WI-19545 | HT3419 | 2984 | calcium channel, voltage- | GAGGCTCCGGGCTGG[C/T]AGGAGGCCTTGATGA | M | C | T | A | V |
| | | | | gated, alpha 1E subunit, | | | | | | |
| | | | | alt. transcript 2, ? | | | | | | |
| G957a30 | WI-19546 | HT3419 | 2989 | calcium channel, voltage- | TCCGGGCTGGCAGGA[G/T]GCCTTGATGAGGCTG | M | G | T | G | C |
| | | | | gated, alpha 1E subunit, | | | | | | |
| | | | | alt. transcript 2, ? | | | | | | |
| G957a31 | WI-19547 | HT3419 | 3000 | calcium channel, voltage- | AGGAGGCCTTGATGA[G/T]GCTGACACCCCCCTA | M | G | T | E | D |
| | | | | gated, alpha 1E subunit, | | | | | | |
| | | | | alt. transcript 2, ? | | | | | | |
| G957a32 | WI-19548 | HT3419 | 3033 | calcium channel, voltage- | CCTGCCCCATCCTGA[G/T]CTGGAAGTGGGGAAG | M | G | T | E | D |
| | | | | gated, alpha 1E subunit, | | | | | | |
| | | | | alt. transcript 2, ? | | | | | | |
| G957a33 | WI-19549 | HT3419 | 4005 | calcium channel, voltage- | CAACTATGTAGATCA[T/C]GAGAAAAACAAGATG | — | T | C | | |
| | | | | gated, alpha 1E subunit, | | | | | | |
| | | | | alt. transcript 2, ? | | | | | | |
| G957a34 | WI-19550 | HT3419 | 5070 | calcium channel, voltage- | AGGGCAGAACGAGAA[C/T]GAACGCTGCGGCACC | — | C | T | | |
| | | | | gated, alpha 1E subunit, | | | | | | |
| | | | | alt. transcript 2, ? | | | | | | |
| G957a35 | WI-19551 | HT3419 | 5808 | calcium channel, voltage- | GAGTGGATACCCTTC[G/A]ATGAGTCCACTCTCT | S | G | A | S | S |
| | | | | gated, alpha 1E subunit, | | | | | | |
| | | | | alt. transcript 2, ? | | | | | | |
| G957a36 | WI-19552 | HT3419 | 5841 | calcium channel, voltage- | CCAGGATATATTCCA[G/A]TTGGCTTGTATGGAC | S | G | A | Q | Q |
| | | | | gated, alpha 1E subunit, | | | | | | |
| | | | | alt. transcript 2, ? | | | | | | |
| G957a37 | WI-19553 | HT3419 | 5860 | calcium channel, voltage- | GCTTGTATGGACCCC[A/G]CCGATGACGGACAGT | — | A | G | | |
| | | | | gated, alpha 1E subunit, | | | | | | |
| | | | | alt, transcript 2, ? | | | | | | |
| G957a38 | WI-19554 | HT3419 | 5922 | calcium channel, voltage- | TAGTGAATTAAAAAG[C/T]GTGCAGCCCTCTAAC | — | C | T | | |
| | | | | gated, alpha 1E subunit, | | | | | | |
| | | | | alt. transcript 2, ? | | | | | | |
| G957a39 | WI-19555 | HT3419 | 6564 | calcium channel, voltage- | ACCTGCTGATGGAAG[C/T]GAGGAGGGCTCCCCG | — | C | T | | |
| | | | | gated, alpha 1E subunit, | | | | | | |
| | | | | alt. transcript 2, ? | | | | | | |
| TBXAS1a33 | WI-19565 | M80647 | 912 | TBXAS1, thromboxane A | GATTTTGCCCAATAA[G/A]AACCGAGACGAACTG | S | G | A | K | K |
| | | | | synthase 1 (platelet, | | | | | | |
| | | | | cytochrome P450, subfamily | | | | | | |
| | | | | V) | | | | | | |
| TEXAS1a34 | WI-19566 | M80647 | 1111 | TBXAS1, thromboxane A | GGGTGCAAGCCGAAC[C/G]CTTCCCGGCAACACC | M | C | G | P | A |
| | | | | synthase 1 (platelet, | | | | | | |
| | | | | cytochrome P450, subfamily | | | | | | |
| | | | | V) | | | | | | |

From the foregoing, it is apparent that the invention includes a number of general uses that can be expressed concisely as follows. The invention provides for the use of any of the nucleic acid segments described above in the diagnosis or monitoring of diseases, such as cancer, inflammation, heart disease, diseases of the cardiovascular system, and infection by microorganisms. The invention further provides for the use of any of the nucleic acid segments in the manufacture of a medicament for the treatment or prophylaxis of such diseases. The invention further provides for the use of any of the DNA segments as a pharmaceutical.

While this invention has been particularly shown and described with references to preferred embodiments thereof, it will be understood by those skilled in the art that various changes in form and details may be made therein without departing from the scope of the invention encompassed by the appended claims.

We claim:

1. A nucleic acid molecule comprising a nucleic acid sequence selected from the group consisting of the nucleic acid sequences listed in the Table, wherein said nucleic acid sequence is at least 10 nucleotides in length and comprises a polymorphic site identified in the Table, and wherein the nucleotide at the polymorphic site is different from a nucleotide at the polymorphic site in a corresponding reference allele.

2. A nucleic acid molecule according to claim 1, wherein said nucleic acid sequence is at least 15 nucleotides in length.

3. A nucleic acid molecule according to claim 1, wherein said nucleic acid sequence is at least 20 nucleotides in length.

4. A nucleic acid molecule according to claim 1, wherein the nucleotide at the polymorphic site is the variant nucleotide for the nucleic acid sequence listed in the Table.

5. An allele-specific oligonucleotide that hybridizes to a portion of a nucleic acid sequence selected from the group consisting of the nucleic acid sequences listed in the Table, wherein said portion is at least 10 nucleotides in length and comprises a polymorphic site identified in the Table, and wherein the nucleotide at the polymorphic site is different from a nucleotide at the polymorphic site in a corresponding reference allele.

6. An allele-specific oligonucleotide according to claim 5 that is a probe.

7. An allele-specific oligonucleotide according to claim 5, wherein a central position of the probe aligns with the polymorphic site of the portion.

8. An allele-specific oligonucleotide according to claim 5 that is a primer.

9. An allele-specific oligonucleotide according to claim 8, wherein the 3′ end of the primer aligns with the polymorphic site of the portion.

10. An isolated gene product encoded by a nucleic acid molecule according to claim 1.

11. A method of analyzing a nucleic acid sample, comprising obtaining the nucleic acid sample from an individual; and determining a base occupying any one of the polymorphic sites shown in the Table.

12. A method according to claim 11, wherein the nucleic acid sample is obtained from a plurality of individuals, and a base occupying one of the polymorphic positions is determined in each of the individuals, and wherein the method further comprising testing each individual for the presence of a disease phenotype, and correlating the presence of the disease phenotype with the base.

13. An oligonucleotide microarray having immobilized thereon a plurality of oligonucleotide probes specific for one or more nucleic acid molecules comprising a nucleic acid sequence selected from the group consisting of the nucleic acid sequences listed in the Table.
